(19) United States
(12) Patent Application Publication
(10) Pub. No.: US 2011/0046056 A1
(43) Pub. Date: Feb. 24, 2011

(54) APOA-1 PEPTIDE MIMETICS

(76) Inventors: Elisabetta Bianchi, Rome (IT); Antonello Pessi, Rome (IT); Raffaele Ingenito, Rome (IT); Jun Wang, Milburn, NJ (US); Ester Carballo-Jane, Atlantic Highlands, NJ (US); Ching H. Chang, Fanwood, NJ (US)

Correspondence Address: MERCK, P O BOX 2000, RAHWAY, NJ, 07065-0907, US

(21) Appl. No.: 12/664,510

(22) PCT Filed: Jun. 18, 2008
(86) PCT No.: PCT/US08/07855

§ 371 Date: Dec. 14, 2009

Related U.S. Application Data
(60) Provisional application No. 60/936,533, filed on Jun. 20, 2007.

(51) Int. Cl. A61K 38/16; C07K 14/00; A61P 9/10

(52) U.S. Cl. 514/7.4; 530/300; 530/326

(57) ABSTRACT

The invention relates to peptide mimetics for treating disorders associated with hypercholesterolemia and cardio-vascular disease. In particular, the invention relates to peptides that mimic the activity of apolipoprotein A-I (Apo AI).

### BACKGROUND OF THE INVENTION

The references cited in the present application are not admitted to be prior art to the claimed invention.

The concentration of cholesterol associated with high-density lipoprotein (HDL) is inversely associated with cardiovascular disease risk (Miller, G. J. and Miller, N. E., Lancet 1 (1975) 16-25). Studies in patients with disorders of HDL metabolism and in genetically modified animals support a relationship between low HDL and the development of atherosclerotic vascular disease. The beneficial effects of HDL is related to its role in mediating transport of cholesterol from peripheral tissues to the liver (reverse cholesterol transport, RCT), where it is eliminated. RCT opposes the action of low-density lipoproteins (LDL) which deliver cholesterol from the liver, where it is synthesized or taken from dietary sources, to peripheral tissues in the body.

The major protein component of HDL, apolipoprotein A-I (ApoA-1), plays a key role in the RCT process. The mechanism of RCT comprises a first step in which free cholesterol is removed from peripheral tissues, including macrophages in the arterial wall. Free cholesterol is then esterified by the action of lecithin:cholesterol acyltransferase (LCAT), exchanged with lower density lipoproteins, transferred to the liver and finally excreted into the bile.

The activity of ApoA-1 requires its interaction with the ATP-binding cassette transporter A1 (ABCA1). ABCA1 mediates the efflux of phospholipids and cholesterol to apolipoprotein acceptors as an initial step in the RCT pathway. Several studies highlight the importance of the ApoA-1-ABCA1 interaction: for example, defective ABCA1 is causal to Tangier disease (Brooks-Wilson, A. et al., Nat. Genet. 22 (1999) 336-344; Bodzioch, M. et al., Nat. Genet. 22 (1999) 347-351), and mutations in ABCA1 abolish the ability of ApoA-1 to mediate phospholipid and cholesterol efflux from cells, causing premature atherosclerosis (Brooks-Wilson, A. et al., Nat. Genet. 22 (1999) 336-344; Bodzioch, M. et al., Nat. Genet. 22 (1999) 347-351; Francis, G. A., J. Clin. Investig. 96 (1995) 78-87; Remaley, A. T. et al., Arterioscler. Throm. Vasc. Biol. 17 (1997) 1813-1821).

Other beneficial effects of HDL include protection of LDL against oxidation, reduction of platelet aggregation, and modulation of endothelial dysfunction and vascular cytokine activation induced by dyslipidemia or atherosclerosis.

### SUMMARY OF THE INVENTION

This invention provides ApoA-1 peptide mimetics. Peptides have been designed to mimic the activity of ApoA-1 in HDL, more specifically in terms of lipid-binding and cholesterol efflux properties via an ABCA-1 dependent pathway. These peptide mimetics are derived from the sequence of the ApoA-1 consensus peptide by mutation of at least one amino acid to a non-natural amino acid, said non-natural amino acid comprising an isobutyric side chain, a dicarboxylic acid side chain, or a hydrocarbon-substituted side chain. Preferred embodiments include ApoA-1 peptide mimetics which exhibit an EC50 between 10-0.4 μM for ABCA 1-dependent cholesterol efflux in the assay(s) described herein

Aspects of this invention are compositions of peptide mimetics and lipid(s). The peptide mimetics are derived from the sequence of the ApoA-1 consensus peptide by mutation of at least one amino acid to a non-natural amino acid, said non-natural amino acid containing an isobutyric side chain, a dicarboxylic acid side chain, or a hydrocarbon-substituted side chain. The compositions of peptide mimetics and lipid(s) can approximate HDL type particles.

Aspects of this invention are pharmaceutical compositions comprising a peptide mimetic and, optionally, one or more lipids in a pharmaceutically acceptable carrier solution. Such compositions should be suitable, for example, in the acute and chronic treatment of atherosclerotic lesions.

Aspects of this invention are methods of increasing reverse cholesterol efflux in a patient comprising administering to the patient a therapeutically effective dose of a peptide mimetic.

Aspects of this invention are methods of increasing reverse cholesterol efflux in a patient comprising administering to the patient a therapeutically effective dose of a peptide mimetic and lipid(s).

Reference to open-ended terms such as “comprises” allows for additional elements or steps. Occasionally phrases such as “one or more” are used with or without open-ended terms to highlight the possibility of additional elements or steps.

Unless explicitly stated, reference to terms such as “a” or “an” is not limited to one. For example, “a cell” does not exclude “cells”. Occasionally phrases such as one or more are used to highlight the possible presence of a plurality.

### BRIEF DESCRIPTION OF THE DRAWINGS

FIG. 1 Structure and helical wheel representation of the Apo A-I consensus sequence (Apo AI_{cons}).

FIG. 2 Representative side chain non-natural amino acids.

FIG. 3 A helical wheel representation of the Structure-Activity Relationship (SAR) strategy pursued.

FIG. 4 The effect of a representative peptide on atheroma volume in ApoE-deficient mice as determined by magnetic resonance imaging (MRI).

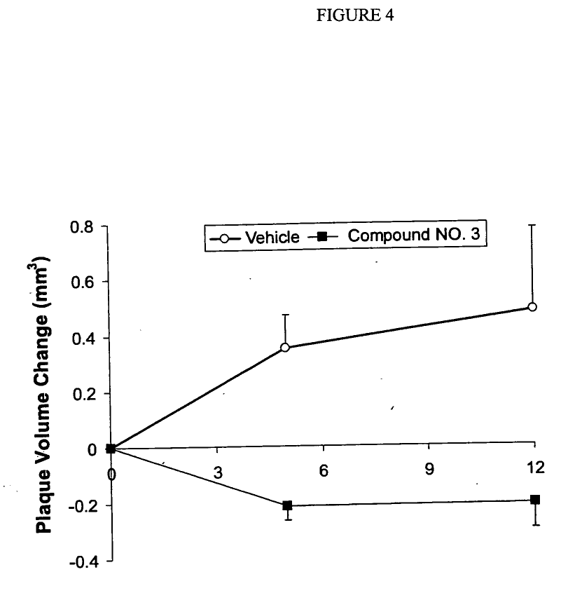

### DETAILED DESCRIPTION OF THE INVENTION

The present invention comprises ApoA-1 peptide mimetics having non-natural amino acids. The peptides promote reverse cholesterol transport and may be useful in the treatment of hypercholesterolemia.

Each HDL particle contains two to four copies of ApoA-1. ApoA-1 is a 243 amino acid protein that consists mainly of 6 to 8 different 22 amino acid repeats spaced by a linker moiety which is often proline. The biological activity of ApoA-1 has been attributed to the presence of these multiple repeats, which display a unique secondary structure element called class A amphipathic α-helix (Segrest, J. P. et al., FEBS Lett. 38 (1974) 247-253). Class A amphipathic helices are characterized by the presence of positively charged amino acid residues at the hydrophilic/hydrophobic interface, while negatively charged residues are clustered at the center of the hydrophilic phase.

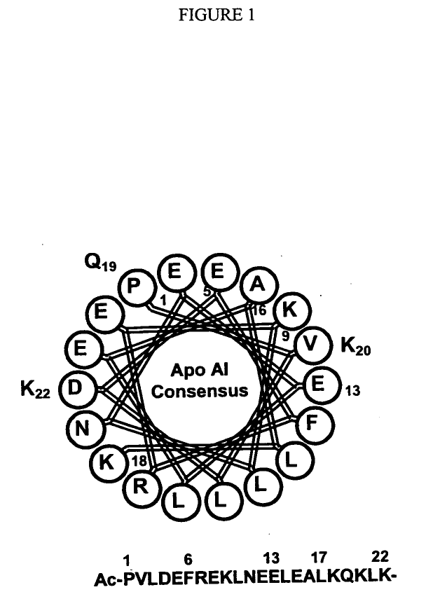

A consensus peptide containing 22-amino acid residues based on the sequence of the helices of human ApoA-1 has been disclosed in the scientific literature (Anantharamaiah, G. M. et al, Arteriosclerosis 10 (1990) 95). The sequence of the consensus peptide (Apo AI_{cons}) was designed by identifying the most prevalent residue at each position of the helical domains of human Apo AI, and the resulting peptide shows the features of a class A amphipathic helix. A helical wheel representation of Apo AI_{cons} is shown in FIG. 1.

However, when Apo AI_{cons} was tested for its ability to clear liposome turbidity and to promote in vitro cholesterol efflux from mouse macrophage cells (see examples of the assays), it was found to be inactive in both assays.

When designing peptide mimetics with desired properties, the particular feature of peptide specificity for cholesterol efflux via the ABCA1 transporter is preferred, especially due to the fact that cholesterol efflux mediated by an ABCA1-independent mechanism might result in cytotoxicity.

Peptides of the present invention are designed by introducing single or multiple mutations at various positions of the Apo AI_{cons} sequence. Peptides are screened for increased membrane binding affinity. Preserving or further stabilizing the class A α-helical conformation of the peptide, which is essential for activity, is also desirable

The introduced mutations were based on non-natural amino acids including α-aminoisobutyric acid (Aib), an amino acid known to stabilize the α-helical secondary structure. In the case of Aib, however, substitution of one Aib residue in position 13 may not be sufficient to produce major improvement in the ability to efflux cholesterol, and this can also be the case for a double mutant with Aib at positions 13 and 16, depending on the position of the mutations.

Improved cholesterol efflux activity can be observed when the Aib mutation is combined with the introduction of γ-carboxyglutamic acid (Gla), an amino acid with an extra carboxylic acid in the side chain with respect to aspartic acid and/or glutamic acid. The double negative charge on Gla effectively increases the local negative charge density in the peptide. For example, the double mutants Gla⁸/Aib¹³ and Gla¹⁵/Aib¹³ show EC₅₀=44 and 41 μM, respectively, in the cholesterol efflux assay. The activity depends on both the Gla and the Aib residues. The corresponding Gla/Ala double mutants at the same positions are less potent in the cholesterol efflux assay. For example, Gla⁸/Ala¹³ and Gla¹⁵/Ala¹³ show EC₅₀=142 and 234 μM, respectively.

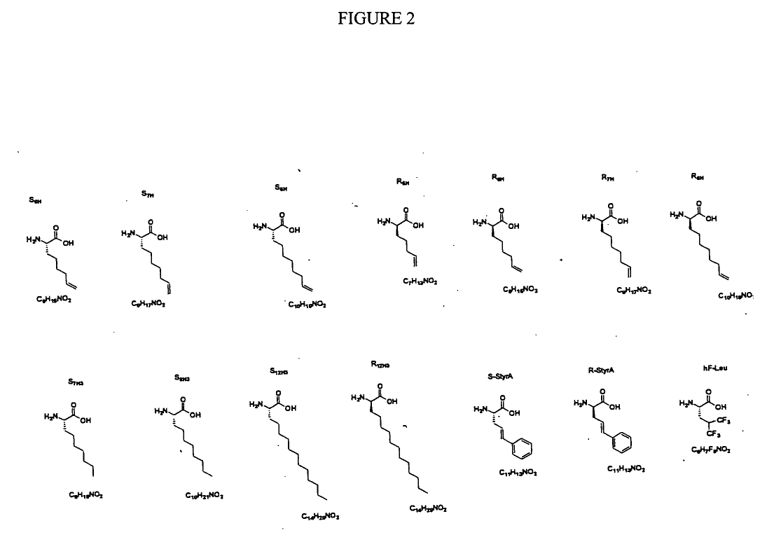

Amino acids containing hydrocarbon-substituted side chains of varying length (S7H3, S8H3 and S12H3 (see also FIG. 2) of both R (or D) and S (or L) stereochemistry, which are previously described by Schafmeister, C. et al (J. Am. Chem. Soc. 122 (2000) 5891, are included within the present invention. In particular, the present invention includes, without limitation such preferred amino acids as (2S)-2-aminonon-8-enoic acid (S7H); (2R)-2-aminonon-8-enoic acid (R7H); (2S)-2-aminooct-7-enoic acid (S6H); (2R)-2-aminooct-7-enoic acid (R6H); (2S)-2-aminononanoic acid (S7H3); an α-, α-disubstituted amino acid such as (2R)-2-amino-2-methylhept-6-enoic acid (R5Me) can be incorporated into ApoA-1 peptide mimetics.

Other mutations which increase the hydrophobic character of the peptide include styryl-alanine (StyrA), a superior analog of phenylalanine, which has an alkenyl linker between the phenyl moiety and the α-carbon; and fluorinated amino acids, such as hexafluoroleucine.

The purpose of introducing single or multiple hydrocarbon-substituted amino acids in Apo AI_{cons} is two-fold. First, such amino acids increase the hydrophobic character of the peptide by providing long alkenyl chains. Second, one can, in certain cases, maintain activity while stabilizing the helical conformation through a ring-closing metathesis of two hydrocarbon-substituted amino acids at positions i, i+4 or i, i+7 of the peptide sequence. Covalent helical stabilization of peptides by ruthenium-catalyzed ring-closing metathesis (RCM) of hydrocarbon-substituted amino acids has been reported (Blackwell, H. E. and Grubbs, R. H., Angew. Chem. Int. Ed. 37 (1998) 3281-3284). Cross-linking based on all-hydrocarbon-substituted amino acids and RCM, which produced a helical cross-link has also been reported (Schafmeister, C. et al., J. Am. Chem. Soc. 122 (2000) 5891; Walensky, L. D. et al., Science 305 (2004) 1466; Bernal, F. et al., J. Am. Chem. Soc. 129 (2007) 2456).

The advantage of a chemically robust, all-hydrocarbon-substituted cross-link to stabilize the peptide α-helical structure is its hydrophobic nature, unlike the other cross-links described for peptides, which are polar (e.g., lactam bridges) or pharmacologically labile (e.g. disulfides). It is therefore particularly suitable for the present invention, in which it is important to preserve both the α-helical structure and a high degree of hydrophobicity for binding to the lipid membrane.

Hydrocarbon-substituted amino acids can be introduced either at a single position or at multiple positions of the Apo AI_{cons}, more particularly, two. When two positions were mutated, the hydrocarbon-substituted amino acid could be the same at both positions, or a different one in each position. Some exemplary peptides are described below.

The hydrocarbon-substituted amino acid, R5H, was introduced at two different positions, K9 and E13, of Apo AIₒₙₛ and another version of this peptide was designed after metathesis of the two R5H groups with formation of the hydrocarbon-substituted bridge. Both peptides promote cholesterol efflux via an ABCA1-dependent mechanism and are active in the liposome solubilization assay. The first peptide showed EC₅₀=46 μM in the cholesterol efflux assay and IC₅₀=91 μM in the liposome solubilization assay. The second peptide with the hydrocarbon-substituted bridge connecting the side chains at position 9 and 13 (i, i+4) is more potent, with EC₅₀=10 μM in cholesterol efflux and IC₅₀=60 μM in the liposome solubilization assay. In this case, formation of the hydrocarbon-substituted bridge led to an increase in activity.

Interestingly, when the same positions K9 and E13 are mutated to S5H, i.e. the hydrocarbon-substituted amino acid with the same side chain length of R5H, but of opposite stereochemistry, S (L) instead of R (D) (FIG. 2), the resulting peptides, one in the open form and one in the closed form after metathesis of the 9/13 S5H side chains, displayed very low to no activity in the cholesterol efflux assay despite good lipid binding.

These data show that for mutations at position K9 and E13 with a C5 hydrocarbon-substituted amino acid, the D configuration (R5H) is preferred over the L-configuration (S5H) for both the open and closed forms of the peptide.

When the same positions K9 and E13 are mutated to RSme, an α,α-di-substituted amino acid with same side chain length of R5H and the same D configuration (FIG. 2), the expectation is that stabilization of the α-helical structure and increased hydrophobicity can translate into higher potency. This was not the case, as both the open and the closed form after metathesis of the 9/13 R5me side chains, displayed very low to no activity in cholesterol efflux despite good lipid binding.

Another peptide in which E13 is mutated to S7H (FIG. 2), was compared with a peptide in which E13 is mutated to R7H (FIG. 2). Both peptides promoted cholesterol efflux by an ABCA1-dependent mechanism and showed activity in the liposome solubilization assay. The peptide with the S7H amino acid at E13 had an EC₅₀=2.8 μM in the cholesterol efflux assay and IC₅₀=11 μM in the liposome solubilization assay while the peptide with the R7H amino acid at E13 had an EC₅₀=7.2 μM in the cholesterol efflux assay and IC₅₀=500 μM in the liposome solubilization assay.

Another peptide in which E13 is mutated to S6H, was compared to a peptide in which E13 is mutated to R6H. Both peptides promoted cholesterol efflux by an ABCA1-dependent mechanism and show activity in the liposome solubilization assay. The peptide with the S6H amino acid at E13 had an EC₅₀=1.4 μM in the cholesterol efflux assay and IC₅₀=500 μM in the liposome solubilization assay while the peptide with the R6H amino acid at E13 had an EC₅₀=18 μM in the cholesterol efflux assay and IC₅₀=32 μM in the liposome solubilization assay.

A peptide in which F6 and E13 (positions i, i+7) are mutated to R6H and S7H, respectively, was compared to the same peptide after metathesis of the R6H and S7H side chains, with formation of the hydrocarbon-substituted bridge. Both peptides promoted cholesterol efflux by an ABCA1-dependent mechanism and show activity in the liposome solubilization assay. The peptide with the R6H amino acid at F6 and the S7H amino acid at E13 had an EC₅₀=0.4 μM in the cholesterol efflux assay and IC₅₀=25 μM in the liposome solubilization assay. The corresponding peptide with the hydrocarbon-substituted bridge connecting the side chain at position 6 and 13 (i, i+7) had an EC₅₀=2.0 μM in cholesterol efflux (ABCA1-dependent) and IC₅₀=43 μM in the liposome solubilization assay. The formation of the hydrocarbon-substituted bridge, in this case, leads to a decrease in activity unlike the result observed for the R5H (i, i+4) peptides.

Inverting the position of the hydrocarbon-substituted amino acids R6H and S7H, i.e., producing the peptide in which S7H is in position F6 and R6H is in position E13 results in a peptide with 2-3-fold lower activity, yielding an EC₅₀=1.2 μM in the cholesterol efflux assay by an ABCA1-dependent mechanism and IC₅₀=44 μM in the liposome solubilization assay

Therefore, one can obtain ApoA-1 peptide mimetics with the desired properties, i.e., capable of promoting cholesterol efflux by an exclusively ABCA1-dependent mechanism from cells with an EC₅₀ in the low micromolar range, or promoting lipid solubilization with an EC₅₀ in the micromolar range, by optimizing the specific position, the specific chemical nature, and the specific chirality of the mutant hydrocarbon-substituted amino acids all need to be simultaneously optimized. Moreover, the presence in the molecule of an all-hydrocarbon-substituted bridge obtained by RCM of the hydrocarbon-substituted amino acid side chains can be beneficial in some cases, and not beneficial in others. Thus the formation of the bridge also needs to be optimized case-by-case.

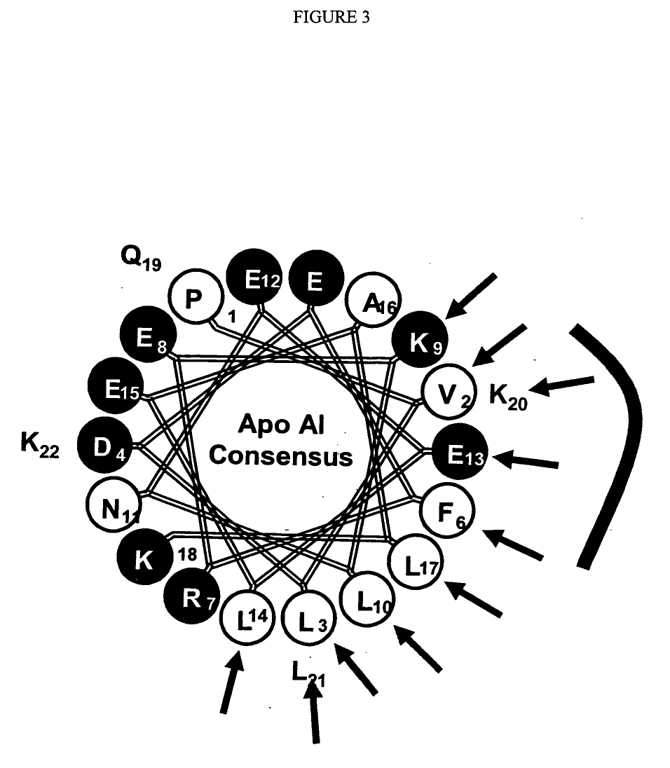

In order to further explore the length and the stereochemistry of non-natural amino acids optimally suitable for the substitution, the Structure-Activity Relationship (SAR) around the hydrophobic face of the α-helix was explored by single point mutations. Hydrocarbon-substituted side chain amino acids of variable length (C5-C12) and different stereochemistry (L or D) were introduced at the positions indicated by the arrows in FIG. 3, which are position 2, 3, 6, 9, 10, 13, 14, 17, 20, and 21 of Apo AI_{cons} sequence.

A series of analogs were synthesized with single point mutations with R6H introduced at positions: 2, 3, 6, 9, 10, 13, 14, 17, 20, 21 of Apo AI_{cons}. The analogs were tested in the in vitro reverse cholesterol transport assay. Only the mutant at position 13 showed activity in the micromolar range with EC₅₀=18 μM.

A series of analogs were synthesized with single point mutations with R7H, the superior homolog of R6H, at position 2, 6, 9, 10, 13, 14, 17, 20, 21 of Apo AI_{cons}. In this case three mutants showed activity in the in vitro RCT assay. While the two mutants with R7H in position 2 and in position 20 were only slightly active, the mutant with R7H in position 13 showed good potency in the micromolar range (EC₅₀=7.2 μM). This mutant was one order magnitude more potent than the R6H.

In summary, when hydrocarbon-substituted amino acids of D-configuration series (R6H, R7H) were introduced as single point mutants, three key positions were identified that have an impact on activity in RCT in vitro, namely position 13, 2 and 20. Interestingly, these three positions are on the same face of the α-helix and occupy a small corridor along the helical axis next to the interface between hydrophobic and positively charged residues (FIG. 3).

Additionally, two single point mutants were obtained by the introduction of the R5H at position 6 and position 13. The peptides were tested in an in vitro RCT assay and showed either no activity or only slight activity (EC₅₀=6.2%@100 μM), suggesting that amino acids with a carbon chain longer than 5 would be required for activity in the in vitro RCT.

The SAR was explored also with hydrocarbon-substituted side chain of L-configuration from 6 carbon chain S6H to 12 carbon chain S12H3. A series of analogs were synthesized with single point mutations with S6H (FIG. 2), at positions: 2, 3, 6, 9, 10, 13, 14, 17, 20, 21 of Apo AI_{cons}. The peptides were tested in the in vitro RCT assay. The single point mutants at position 2 and at position 20 were only slightly active. The mutant with S6H at position 13 showed a potency in the micromolar range superior to its R6H analog (EC₅₀=1.4 μM). This finding indicates that the substitution at the position 13 is very important to confer activity from the Apo AI_{cons} and that the L-configuration is preferred.

Another series of analogs were synthesized with single point mutations with S7H (FIG. 2), the superior homolog of S6H, at position 6, 9, 10, 13, 14, 17, of Apo AI_{cons}. The peptides were tested in the in vitro RCT assay. The mutant with S7H at position 6 showed an EC₅₀=4.0%@100 μM while the mutant with S7H at position 13 was more potent and showed an EC₅₀=2.0 μM. These data further confirm the finding that introduction of a long alkenyl chain such as S7H, a 7 carbon chain at position 13, has a great impact in the gain of activity in the in vitro RCT assay.

Further mutations at position 13 were then explored. A peptide with S8H, the superior homolog of S7H, at position 13 of Apo AI_{cons} was synthesized. The peptide showed an EC₅₀=0.6 μM in the RCT in vitro assay, supporting the notion that elongation of the alkenyl chain from 7 carbons to 8 carbons brings a gain in activity.

In summary, when hydrocarbon-substituted amino acids of L-configuration series (S6H, S7H, S8H) were introduced as single point mutant on the Apo AI_{cons}, four key positions were identified to have an impact on activity in the RCT in vitro assay, namely, positions 13, 6, 2 and 20. Interestingly, these four positions, as found for the SAR exploration done with hydrocarbon-substituted amino acids of D-configuration, are on the same face of the α-helix and occupy a small corridor along the helical axis next to the interface between hydrophobic and positively charged residues (FIG. 3).

In addition to the introduction of hydrocarbon-substituted non-natural amino acids, the SAR around the hydrophobic face of the α-helix of Apo AI_{cons} was explored also with alkyl side chain amino acids of both L- and D-configuration. Single point mutants were obtained when S7H3, S8H3 S12H3 or R12H3 (FIG. 2) were introduced either in position 6 or 13 of the Apo AI_{cons} sequence. The mutants obtained when S7H3 and S8H3 were introduced at position 13 showed very good potency in the RCT in vitro assay with EC₅₀=0.6 μM and EC₅₀=0.3 μM, respectively. As shown by the data these two saturated analogs were somewhat more active than the corresponding unsaturated peptides. When longer alkyl side chains, (RS)12H3, were introduced at positions 6 or 13, the corresponding single point mutants showed EC₅₀=0.1-0.4 μM or EC₅₀=0.2-0.4 μM.

Thus it is apparent that in the case of position 13, the elongation from 8 carbon chain to 12 carbon chain does not confer much gain in activity while for position 6, a considerable gain in activity is achieved from the 7 carbon alkenyl chain (S7H3) to the 12 carbon alkyl chain (RS12H3).

The SAR was further explored with Styryl-Alanine (StyrA), a superior analog of phenylalanine with an alkenyl linker between the phenyl moiety and the α-carbon. Styryl-Alanine further increases the hydrophobic character of the peptide. The single mutants obtained introducing (D)-StyrA and (L)-StyrA at position 13 showed EC₅₀=22 μM and EC₅₀=1 μM respectively in the RCT in vitro assay. This suggested that the introduction of a hydrophobic/aromatic amino acid at position 13 is quite beneficial for gaining activity in the in vitro RCT assay and that the L-configuration is preferred. The single mutants obtained introducing (D)-StyrA and (L)-StyrA at position 6 were inactive in the RCT in vitro assay.

L-hexafluoro-Leucine (hF-L-Leu) (FIG. 2), a hydrophobic amino acid which was introduced in order to increase the hydrophobic character while stabilizing the α-helical conformation of ApoAI_{cons} sequence, showed potency in the micromolar range. The single point mutant obtained introducing the hF-L-Leu at position 13 showed an EC₅₀=1.1 μM in the RCT in vitro assay. This finding confirms that hydrophobic amino acids at position 13 are beneficial for increasing RCT in the in vitro assay.

To summarize the SAR analysis done with single point mutants with hydrocarbon-substituted amino acids of both D-configuration and L-configuration of different length (C5-C12), the three main findings are that substitution at positions 2, 6, 13 and 20 gained potency from the inactive Apo AI_{cons} sequence; position 13 was identified as the most important of the four; and that the L-configuration was preferred.

Based on these findings, it was decided to explore the SAR with a combination of mutations, and introduce double or multiple mutations in the same peptide sequence. Hydrocarbon-substituted side chain amino acids were introduced either at two positions or at multiple positions (up to 5) of the Apo AI_{cons}. When two or more positions were mutated at the same time, the hydrocarbon-substituted side chain amino acids could be the same at any position or different in each position.

Double mutants were designed with S7H at position 2 and 13 (EC₅₀=0.3 μM) and at position 6 and 13 (EC₅₀=0.3 μM) and at position 13 and 20 (EC₅₀=0.6 μM) or as a combination of (L)-StyrA at position 6 and S7H at position 13 (EC₅₀=0.2 μM), thus showing that the combination of two mutations at key positions produced an additive effect on the potency in RCT in vitro assay.

Furthermore, two other mutants were designed with S7H at position 13 and γ-carboxyglutamic acid (Gla), an amino acid with an extra carboxylic acid in the side chain, either at position 8 or 15. Gla was introduced to increase the local negative charge density in the peptide. The two double mutants Gla⁸/S_{7H}¹³ and S_{7H}¹³/Gla¹⁵ showed EC₅₀=0.9 μM and 0.7 μM in the RCT in vitro assay respectively. The activity depends on both the Gla and the S_{7H} residues, as the corresponding Glu^{8&15}/S_{7H}¹³ mutant (EC₅₀=1.6 μM) is less potent, thus confirming that the proper combination of two mutations produced an additive effect on the potency.

When S8H was introduced in position 6 and 13 (EC₅₀=0.4 μM) the peptide showed potency superior to that observed for the single mutant (EC₅₀=0.6 μM). This potency was similar to that of the double mutant with S7H (EC₅₀=0.3 μM), indicating that elongation of carbon chain amino acids (>S7H) is no longer beneficial for double mutants (position 6 and 13) for the RCT in vitro activity.

Double mutants were also designed by introduction of the alkyl side-chain amino acids. When S7H3 was introduced at positions 6 and 13, the peptide was more potent than the corresponding single mutant with an EC₅₀=0.3 μM. The double mutant with S8H3 showed an EC₅₀=0.6 μM, thus confirming that the double mutant peptide with longer carbon chain amino acids (C8) does not show increased activity in the RCT assay with respect to the double mutant with C7 amino acids.

The SAR was further analyzed with the introduction of multiple mutations in the same peptide sequence as described above, with no further improvement on the potency (ECₛₒ from 7.7 μM to 0.1 μM).

In addition to making amino acids substitutions, other methods were used to obtain peptides with desired properties, i.e., specificity for cholesterol efflux via the ABCA1 transporter. For example, elongation of the sequence to form either dimeric peptides with no spacer (EC50=41 μM) or dimeric peptides by disulfide bridge formation (EC50=1.5 μM) with one or more amino acid substitutions.

Additional approaches included, for example, the replacement of Arg with Lys residue in position 7 or replacement of Asp and Asn residues with Glu and Gln respectively, to improve either the chemical synthesis or the stability of peptides without losing activity in the RCT in vitro assay.

Furthermore, additional attempts to improve the pharmacokinetic properties of peptides, such as half-life, clearance, exposure, excretion profile include:

(i) conjugation of peptides with a PEG moiety;

(ii) conjugation of peptides with cholesterol;

(iii) replacement of L-amino acids with identical D-amino acids in the sequence; and

(iv) formulation with phospholipids.

The peptides in Tables 1 and 2, some of which display an EC₅₀ as low as ˜0.1 μM for ABCA1-dependent cholesterol efflux, represent novel ApoA-1 mimetics, whose design could not be derived from prior art.

TABLE 1
Sequence of Exemplary ApoA-1 Peptide Mimetics.

| SEQ ID | | |
|---|---|---|
| NO | PEPTIDE | SEQUENCE¹ |
| 1 | CONS | PVLDEFREKLNEELEALKQKLK |
| 2 | GLA8/AIB13 | PVLDEFRGLAKLNEAIBLEALKQKLK |
| 3 | GLA8/ALA13 | PVLDEFRGLAKLNEALEALKQKLK |
| 4 | GLA15/AIB13 | PVLDEFREKLNEAIBLGLAALKQKLK |
| 5 | GLA15/ALA13 | PVLDEFREKLNEALGLAALKQKLK |
| 6 | AIB13 | PVLDEFREKLNEAIBLEALKQKLK |
| 7 | AIB13/AIB16 | PVLDEFREKLNEAIBLEAIBLKQKLK |
| 8 | R5H [E13-L17]R5H | PVLDEFREKLNER_{5H}LEAR_{5H}KQKLK |
| | OPEN | |
| 9 | R5H [K9-E13]R5H | PVLDEFRER_{5H}LNER_{5H}LEALKQKLK |
| | OPEN | |
| 10 | S7H [F6-E13]R6H | PVLDES_{7H}REKLNER_{6H}LEALKQKLK |
| | OPEN | |
| 11 | R5H [E13-L17]R5H | PVLDEFREKLNER_{5H}LEAR_{5H}KQKLK |
| | CLOSED RING | (CYCLO R5H₁₃-R5H₁₇) |
| 12 | R5H [K9-E13]R5H | PVLDEFRER_{5H}LNER_{5H}LEALKQKLK |
| | CLOSED RING | (CYCLO R5H₉-R5H₁₃) |
| 13 | R6H [F6-E13]S7H | PVLDER_{6H}REKLNES_{7H}LEALKQKLK |
| | OPEN | |
| 14 | R6H [F6-E13]S7 | PVLDER_{6H}REKLNES_{7H}LEALKQKLK |
| | CLOSED RING | (CYCLO R6H₆-S7H₁₃) |
| 15 | S5H [K9-E13]S5H | PVLDEFRES_{5H}LNES_{5H}LEALKQKLK |
| | OPEN | |
| 16 | R5ME [K9-E13]R5ME | PVLDEFRER_{5ME}LNER_{5ME}LEALKQKLK |
| | OPEN | |
| 17 | CONS[R_{5H}¹³] | PVLDEFREKLNER_{5H}LEALKQKLK |
| 18 | CONS[R_{6H}¹³] | PVLDEFREKLNER_{6H}LEALKQKLK |
| 19 | CONS[R_{6H}²⁰] | PVLDEFREKLNEELEALKQR_{6H}LK |
| 20 | CONS[R_{7H}²] | PR_{7H}LDEFREKLNEELEALKQKLK |
| 21 | CONS[R_{7H}¹³] | PVLDEFREKLNER_{7H}LEALKQKLK |
| 22 | CONS[R_{7H}²⁰] | PVLDEFREKLNEELEALKQR_{7H}LK |
| 23 | CONS[S_{6H}²] | PS_{6H}LDEFREKLNEELEALKQKLK |
| 24 | CONS[S_{6H}¹³] | PVLDEFREKLNES_{6H}LEALKQKLK |
| 25 | CONS[S_{6H}²⁰] | PVLDEFREKLNEELEALKQS_{6H}LK |
| 26 | CONS[S_{7H}⁶] | PVLDES_{7H}REKLNEELEALKQKLK |
| 27 | CONS[S_{7H}⁹] | PVLDEFRES_{7H}LNEELEALKQKLK |
| 28 | CONS[S_{7H}¹⁰] | PVLDEFREKS_{7H}NEELEALKQKLK |
| 29 | CONS[S_{7H}¹³] | PVLDEFREKLNES_{7H}LEALKQKLK |
| 30 | CONS[S_{7H}¹⁷] | PVLDEFREKLNEELEAS_{7H}KQKLK |
| 31 | CONS[K⁷, S_{7H}¹³] | PVLDEFKEKLNES_{7H}LEALKQKLK |
| 32 | CONS[S_{8H}¹³] | PVLDEFREKLNES_{8H}LEALKQKLK |
| 33 | CONS[E⁴, Q^{11,} S_{8H}¹³] | PVLEEFREKLQES_{8H}LEALKQKLK |
| 34 | CONS[S_{7H3}¹³] | PVLDEFREKLNES_{7H3}LEALKQKLK |
| 35 | CONS[S_{8H3}¹³] | PVLDEFREKLNES_{8H3}LEALKQKLK |
| 36 | CONS[E⁴, Q^{11,} | PVLEEFREKLQES_{8H3}LEALKQKLK |
| | S_{8H3}¹³,] | |
| 37 | CONS[(RS)_{12H3}⁶] | PVLDE-(RS)_{12H3}-REKLNEELEALKQKLK |
| 38 | CONS[(RS)_{12H3}¹³] | PVLDEFREKLNE-(RS)_{12H3}-LEALKQKLK |
| 39 | CONS[(R)STYRA¹³] | PVLDEFREKLNE-(R)STYRA-LEALKQKLK |
| 40 | CONS[(S)STYRA¹³] | PVLDEFREKLNE-(S)STYRA-LEALKQKLK |
| 41 | CONS[HF-LEU^{,13}] | PVLDEFREKLNE-HFLEU-LEALKQKLK |
| 42 | CONS[S_{7H}^{2,13}] | PS_{7H}LDEFREKLNES_{7H}LEALKQKLK |
| 43 | CONS[S_{7H}^{6,13}] | PVLDES_{7H}REKLNES_{7H}LEALKQKLK |
| 44 | CONS[S_{7H}^{13,20}] | PVLDEFREKLNES_{7H}LEALKQS_{7H}LK |
| 45 | CONS[E⁴, S_{7H}^{6,13}, | PVLEES_{7H}REKLQES_{7H}LEALKQKLK |
| | Q¹¹] | |
| 46 | CONS[GLA⁸, S_{7H}¹³] | PVLDEFR-GLA-KLNES_{7H}LEALKQKLK |
| 47 | CONS[S_{7H}¹³, GLA¹⁵] | PVLDEFREKLNES_{7H}L-GLA-ALKQKLK |
| 48 | CON[(S)STYRA⁶, | PVLDE-(S)STYRA-REKLNES_{7H}LEALKQKLK |
| | S_{7H}¹³] | |
| 49 | CONS[S_{8H}^{6,13}] | PVLDES_{8H}REKLNES_{8H}LEALKQKLK |
| 50 | CONS[S_{7H3}^{6,13}] | PVLDES_{7H3}REKLNES_{7H3}LEALKQKLK |
| 51 | CONS[S_{8H3}^{6,13}] | PVLDES_{8H3}REKLNES_{8H3}LEALKQKLK |
| 52 | CONS[S_{7H}^{2,6,13}] | PS_{7H}LDES_{7H}REKLNES_{7H}LEALKQKLK |
| 53 | CONS[S_{7H}^{2,13,20}] | PS_{7H}LDEFREKLNES_{7H}LEALKQS_{7H}LK |
| 54 | CONS[S_{7H}^{6,13,20}] | PVLDES_{7H}REKLNES_{7H}LEALKQS_{7H}LK |
| 55 | CONS[R_{7H}⁶, S_{7H}^{13,20}] | PVLDER_{7H}REKLNES_{7H}LEALKQS_{7H}LK |
| 56 | CONS[GLA¹⁵, | P-S_{7H}-LDEFREKLNES_{7H}L-GLA-ALKQS_{7H}LK |
| | S_{7H}^{2,13,20}] | |
| 57 | CONS[GLA¹⁵, | PVLDES_{7H}REKLNES_{7H}L-GLA-ALKQS_{7H}LK |
| | S_{7H}^{6,13,20}] | |
| 58 | CONS[R_{7H}⁶, S_{7H}^{2,13}] | PS_{7H}LDER_{7H}REKLNES_{7H}LEALKQKLK |
| 59 | CONS[GLA¹⁵, | PS_{7H}LDES_{7H}REKLNES_{7H}L-GLA-ALKQKLK |
| | S_{7H}^{2,6,13}] | |
| 60 | CONS[GLA¹⁵, R_{7H}⁶, | P-S_{7H}LDER_{7H}REKLNE-S_{7H}L-GLA-ALKQKLK |
| | S_{7H}^{2,13}] | |
| 61 | CONS[S_{7H}^{2,6,13,20}] | PS_{7H}LDES_{7H}REKLNES_{7H}LEALKQS_{7H}LK |
| 62 | CONS[S_{7H}^{2,6,13,20}, | PS_{7H}LDES_{7H}REKLNES_{7H}L-GLA-ALKQS_{7H}LK |
| | GLA¹⁵] | |
| 63 | CONS[S_{7H}¹³]₂ | PVLDEFREKLNES_{7H}LEALKQKLKPVLDEFREKLNE-S_{7H}-LEALKQKLK |
| 64 | (CONS[S_{7H}^{6,13}]-GC)₂ | (PVLDES_{7H}REKLNES_{7H}LEALKQKLKGC)₂ |
| 65 | CONS[S_{7H}^{6,13}]-GC- | PVLDES_{7H}REKLNES_{7H}LEALKQKLKGC(PEG₄₀) |
| | PEG₄₀ | |
| 66 | CONS[S_{7H}^{6,13}]-GC- | PVLDES_{7H}REKLNES_{7H}LEALKQKLKGC((MA-OXA12- |
| | OXA₁₂- | GLY_CHOLESTEROL) |
| | CHOLESTEROL | |
| 67 | D-CONS[S_{6H}⁶, R_{7H}¹³] | PVLDES_{6H}REKLNER_{7H}LEALKQKLK |
| 68 | D-CONS[R_{7H}^{6,13}] | PVLDER_{7H}REKLNER_{7H}LEALKQKLK |
| 69 | D-CONS[S_{7H}^{6,13}] | PVLDES_{7H}REKLNES_{7H}LEALKQKLK |
| 70 | D-CONS[R_{7H}²] | PR_{7H}LDEFREKLNEELEALKQKLK |
| 71 | D-CONS[R_{7H}⁶] | PVLDER_{7H}REKLNEELEALKQKLK |
| 72 | D-CONS[R_{7H}¹³] | PVLDEFREKLNER_{7H}LEALKQKLK |

TABLE 2

| | | | ABCA1- |
|---|---|---|---|
| | | ¹LIPID | DEPENDENT |
| | | SOLUBILI- | CHOLESTEROL |
| SEQ ID | | ZATION | EFFLUX |
| NO | PEPTIDE | IC₅₀ (MM) | EC₅₀ (MM) |
| 1 | CONS | NOT ACTIVE | NOT ACTIVE |
| 2 | GLA8/AIB13 | 3744 | 44 |
| 3 | GLA8/ALA13 | NOT ACTIVE | 142 |
| 4 | GLA15/AIB13 | 4000 | 41 |
| 5 | GLA15/ALA13 | 4200 | 234 |
| 6 | AIB13 | 348 | 6% AT 100 UM |
| 7 | AIB13/AIB16 | 1000 | 5% AT 150 UM |
| 8 | R5H [E13-L17]R5H OPEN | 511 | <3% AT 30 UM |
| 9 | R5H [K9-E13]R5H OPEN | 91 | 46 UM |
| 10 | S7H [F6-E13]R6H OPEN | 44 | 1.3 UM |
| 11 | R5H [E13-L17]R5H | 422 | <2% AT 30 UM |
| | CLOSED RING | | |
| 12 | R5H [K9-E13]R5H CLOSED | 68 | 10 UM |
| | RING | | |
| 13 | R6H [F6-E13]S7H OPEN | 25 | 0.4 |
| 14 | R6H [F6-E13]S7 | 43 | 2 |
| | CLOSED RING | | |
| 15 | S5H [K9-E13]S5H OPEN | 15 | 4% @ 10 |
| 16 | R5ME [K9-E13]R5ME OPEN | 24 UM | 6% @ 49 |
| 17 | CONS[R_{5H}¹³] | 500 | 6.2% @ 100 |
| 18 | CONS[R_{6H}¹³] | 32 | 18 |
| 19 | CONS[R_{6H}²⁰] | 32 | 1% @ 100 |
| 20 | CONS[R_{7H}²] | 500 | 6.3% @ 100 |
| 21 | CONS[R_{7H}¹³] | 500 | 7.2 |
| 22 | CONS[R_{7H}²⁰] | 479 | 1.4% @ 100 |
| 23 | CONS[S_{6H}²] | 500 | 1% @ 100 |
| 24 | CONS[S_{6H}¹³] | 500 | 1.4 |
| 25 | CONS[S_{6H}²⁰] | 500 | 236 |
| 26 | CONS[S_{7H}⁶] | 214 | 4% @ 100 |
| 27 | CONS[S_{7H}⁹] | 27 | 1.4% @ 100 |
| 28 | CONS[S_{7H}¹⁰] | 102 | 1.4% @ 100 |
| 29 | CONS[S_{7H}¹³] | 11 | 2 |
| 30 | CONS[S_{7H}¹⁷] | 108 | 1% @ 100 |
| 31 | CONS[K⁷, S_{7H}¹³] | 116 | 1.3 |
| 32 | CONS[S_{8H}¹³] | 500 | 0.6 |
| 33 | CONS[E⁴, Q^{11,} S_{8H}¹³] | 44 | 0.3 |
| 34 | CONS[S_{7H3}¹³] | 429 | 0.6 |
| 35 | CONS[S_{8H3}¹³] | 500 | 0.3 |
| 36 | CONS[E⁴, Q^{11,} S_{8H3}¹³,] | 55 | 0.2 |
| 37 | CONS[(RS)_{12H3}⁶] | 500-500 | 0.2-0.4 |
| 38 | CONS[(RS)_{12H3}¹³] | 500-500 | 0.1-0.4 |
| 39 | CONS[(R)STYRA¹³] | 500 | 22 |
| 40 | CONS[(S)STYRA¹³] | 500 | 1 |
| 41 | CONS[HF-LEU^{,13}] | 68 | 1.1 |
| 42 | CONS[S_{7H}^{2,13}] | 500 | 0.3 |
| 43 | CONS[S_{7H}^{6,13}] | 500 | 0.3 |
| 44 | CONS[S_{7H}^{13,20}] | 500 | 0.6 |
| 45 | CONS[E⁴, S_{7H}^{6,13}, Q¹¹] | 500 | 0.7 |
| 46 | CONS[GLA⁸, S_{7H}¹³] | 500 | 0.9 |
| 47 | CONS[S_{7H}¹³, GLA¹⁵] | 500 | 0.7 |
| 48 | CON[(S)STYRA⁶, S_{7H}¹³] | 500 | 0.2 |
| 49 | CONS[S_{8H}^{6,13}] | 34 | 0.4 |
| 50 | CONS[S_{7H3}^{6,13}] | 500 | 0.3 |
| 51 | CONS[S_{8H3}^{6,13}] | 500 | 0.6 |
| 52 | CONS[S_{7H}^{2,6,13}] | 142 | 0.1 |
| 53 | CONS[S_{7H}^{2,13,20}] | 500 | 3.2 |
| 54 | CONS[S_{7H}^{6,13,20}] | 500 | 0.2 |
| 55 | CONS[R_{7H}⁶, S_{7H}^{13,20}] | 500 | 0.1 |
| 56 | CONS[GLA¹⁵, S_{7H}^{2,13,20}] | 500 | 7.7 |
| 57 | CONS[GLA¹⁵, S_{7H}^{6,13,20}] | 500 | 0.5 |
| 58 | CONS[R_{7H}⁶, S_{7H}^{2,13}] | 42 | 0.2 |
| 59 | CONS[GLA¹⁵, S_{7H}^{2,6,13}] | 500 | 0.6 |
| 60 | CONS[GLA¹⁵, R_{7H}⁶, S_{7H}^{2,13}] | 63 | 0.3 |
| 61 | CONS[S_{7H}^{2,6,13,20}] | 500 | 2.0 |
| 62 | CONS[S_{7H}^{2,6,13,20}, GLA¹⁵] | 500 | 2.4 |
| 63 | CONS[S_{7H}¹³]₂ | 500 | 41 |
| 64 | (CONS[S_{7H}^{6,13}]-GC)₂ | 500 | 1.5 |
| 65 | CONS[S_{7H}^{6,13}]-GC-PEG₄₀ | 400 | 1% @ 100 |
| 66 | CONS[S_{7H}^{6,13}]-GC-OXA₁₂- | 500 | 1.9% @ 30 |
| | CHOLESTEROL | | |
| 67 | D-CONS[S_{6H}⁶, R_{7H}¹³] | 61 | 1.2 |
| 68 | D-CONS[R_{7H}^{6,13}] | 138 | 17.7% @ 100 |
| 69 | D-CONS[S_{7H}^{6,13}] | 15 | 3.0 |
| 70 | D-CONS[R_{7H}²] | 167 | 31.3% @ 100 |
| 71 | D-CONS[R_{7H}⁶] | 408 | 19% @ 100 |
| 72 | D-CONS[R_{7H}¹³] | 114 | 1.1 |

¹Measured at 2 h

The ApoA-1 peptide mimetics of the present invention are proposed for treating disorders associated with hypercholesterolemia and cardiovascular disease. In particular, these peptides mimic the activity of ApoA-1, more specifically in its lipid-binding and cholesterol efflux capabilities via an ABCA-1 dependent pathway, and hence should be suitable for the acute and chronic treatment of atherosclerotic lesions.

The ApoA-1 peptide mimetics of the present invention can be synthesized or manufactured using any technique known in the art. The peptides can be prepared with capped termini by methods commonly used in the art such as N-terminal acetyl and/or C-terminal carboxyamide capping groups. The peptides can be prepared as any pharmaceutically acceptable salt and acetate salts are exemplified herein. ApoA-1 peptide mimetics can be stored in a stabilized form through lyophilization in any convenient amount. The ApoA-1 peptide mimetics can be reconstituted by rehydration with sterile water or an appropriate sterile buffered solution prior to administration to a patient. The ApoA-1 peptide mimetics can be formulated with pharmaceutically suitable excipients.

In certain embodiments, it may be preferred to formulate and administer the ApoA-1 peptide mimetics in a peptide-lipid complex. Formulating the ApoA-1 peptide mimetics of this invention with lipids can be done, for example, by co-lyophilizing the mimetic with a lipid to form a mixture that can be reconstituted into a sterile peptide mimetic/lipid complex. Exemplary techniques are well-known to the skilled artisan. Although any suitable lipid may be employed, a preferred embodiment is 1-palmitoyl-2-linoleoyl phosphatidylcholine (PLPC).

Formulating peptide mimetics with lipids has several advantages since the complex could have an increased half-life in the circulation, particularly when the complex has a similar size and density to the HDL class of proteins, especially the pre-beta HDL populations. The HDL class of lipoproteins can be divided into a number of subclasses based on such characteristics as size, density and electrophoretic mobility. Some examples, in order of increasing size are micellar pre-beta HDL of diameter 50 to 60 Angstroms, discoidal HDL of intermediate size i.e., with a mass of 65 kDa (about 70 Angstroms), spherical HDL3 or HDL2 of diameter 90 to 120 Angstroms. (J. Kane, 1996 in V. Fuster, R. Ross and E. Topol [eds.] Atherosclerosis and Coronary Artery Disease, p. 99; A. Tall and J. Breslow, ibid., p. 106; Barrans et al., Biochemica et Biophysica Acta1300, p. 73-85; and Fielding et al., 1995, J. Lipid Res36, p. 211-228). However, peptide mimetic-lipid complexes of smaller or larger size than HDL may also be formed by the peptide mimetics of the invention.

The peptide mimetic-lipid complexes can conveniently be prepared as stable preparations, having a long shelf life, by a co-lyophilization procedure described below. The lyophilized peptide mimetic-lipid complexes can be used to prepare bulk drug material for pharmaceutical reformulation, or to prepare individual aliquots or dosage units which can be reconstituted by rehydration with sterile water or an appropriate buffered solution prior to administration to a subject.

A simple method for preparing peptide-(phospho)lipid complexes which have characteristics similar to HDL involves combining the peptide mimetic and lipid in a solvent system which co-solubilizes each ingredient. The solvent pairs must be carefully selected to ensure co-solubility of both the peptide mimetic and the lipid. In one exemplary method, the peptide mimetic(s) to be incorporated into the particles can be dissolved in an aqueous or organic solvent or mixture of solvents (solvent 1). The (phospho)lipid component is dissolved in an aqueous or organic solvent or mixture of solvents (solvent 2) which is miscible with solvent 1, and the two solutions are combined. Alternatively, in another exemplary method, the (phospho)lipid component is dissolved directly in the peptide mimetic solution. Alternatively, the peptide mimetic and lipid can be incorporated into a co-solvent system, i.e., a mixture of the miscible solvents. Depending on the lipid binding properties of the peptide mimetic, those skilled in the art will recognize that enhanced or even complete solubilization (and/or enhanced mixing) may be necessary prior to lyophilization; thus, the solvents can be chosen accordingly.

In this method a suitable proportion of peptide mimetic to lipids is first determined empirically so that the resulting complexes possess the appropriate physical and chemical properties. Appropriate properties can include, usually but not always, similarity in size to HDL2 or HDL3. The lipid to peptide mimetic molar ratio should be in the range of about 2 to about 200, and preferably 5 to 50 depending on the desired type of complexes. Examples of such size classes of peptide mimetic-lipid complexes include, but are not limited to, micellar or discoidal particles (usually smaller than HDL3 or HDL2), spherical particles of similar size to HDL2 or HDL3 and larger complexes which are larger than HDL2. [See Bakogianni et al., Diabetes Complications. 2001 September-October; 15(5):265-9 for nomenclature conventions]

Gel filtration chromatography can be used to assess the size of the complexes of lipid and peptide mimetic, for example, the following columns might be used: Pharmacia Superose 6, Pharmacia Superdex 200HR, Phenomenex BioSep SEC S 2000 HPLC. An eluant appropriately contains 100 mM NaCl in an appropriate buffer. A typical sample volume is 10 to 200 microliters of complexes containing 0.5 mM mg peptide mimetic. The column flow rate can appropriately be 0.3 ml/min. A series of proteins of known molecular weight and Stokes' diameter as well as human HDL are used as standards to calibrate the column. The proteins and lipoprotein complexes are monitored by absorbance or scattering of light of wavelength 220 or 280 nm. An example of an HDL which can be used as a standard during chromatography are mature HDL2 particles. Pre-β1 HDL are micellar complexes of apolipoprotein and few molecules of phospholipids. Pre-β2 HDL are discoidal complexes of apolipoprotein and molecules of phospholipids. The more lipids (triglycerides, cholesterol, phospholipids) are incorporated the bigger will become the HDL and its shape is modified. (Pre-β1 HDL (micellar complex)→-Pre-β2 HDL (discoidal complex))→HDL3 (spherical complex)→HDL2 (spherical complex).

Once the solvent is chosen and the peptide mimetic and lipid have been incorporated, the resulting mixture is lyophilized. An additional solvent can be added to the mixture to facilitate lyophilization if desired. The lyophilized product can be stored for long periods and will remain stable.

The lyophilized complexes can be reconstituted in order to obtain a solution or suspension of the peptide mimetic-lipid complex. To this end, the lyophilized powder may be rehydrated with an aqueous solution to a suitable volume (often about 5 mg peptide/ml which is convenient for intravenous injection). In an exemplary embodiment, the lyophilized powder is rehydrated with phosphate buffered saline or a physiological saline solution. The mixture may require agitation to facilitate rehydration. The reconstitution is typically conducted at a temperature equal to or greater than the phase transition temperature (Tm) of the lipid component of the complexes. Within minutes, a solution of reconstituted lipid-peptide mimetic complexes results. The solution may be clear if the complexes are small.

The solvents that may be used include but are not limited to nonpolar, polar, aprotic, and protic organic solvents and the like such as ethanol, methanol, cyclohexane, 1-butanol, isopropyl alcohol, xylene, THF, ether, methylene chloride benzene and chloroform. One can also use solvent mixtures as well as single solvents. Further, prior to use the organic solvents maybe dried to remove water; however, hydrated solvents or water may be used with certain lipids or peptide mimetics. Water may be a suitable solvent, or hydrated solvents or organic solvent/water mixtures may be used, however, if water is used it must be detergent free. The solvents are preferably of the purest quality and the solvents should be salt free and free of particulates. However, the solvents need not be sterile as the resulting product can be sterilized before, during or after lyophilization, in accordance with known techniques in the pharmaceutical art, such as those described in Remington's Pharmaceutical Sciences, 16th and 18th Eds., Mack Publishing Co., Easton, Pa. (1980 and 1990), herein incorporated by reference in its entirety, and in the United States Pharmacopeia/National Formulary (USP/NF) XVII, herein incorporated by reference in its entirety.

The lipids which may be used include but are not limited to natural and synthesized (synthetic) lipids and phospholipids including small alkyl chain phospholipids, egg phosphatidylcholine, soybean phosphatidylcholine, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, sphingomyelin sphingolipids, phosphatidylglycerol, diphosphatidylglycerol dimyristoylphosphatidylglycerol, dipalmitoylphosphatidylglycerol, distearoylphosphatidylglycerol, dioleoylphosphatidylglycerol, dimyristoylphosphatidic acid dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, brain sphingomyelin, dipalmitoylsphingomyelin, distearoylsphingomyelin, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio) hexyl ether glycolipids, and cholesterol and its derivatives.

Administration of the pharmaceutical compositions as described above to a patient in need thereof, can be carried out using known procedures at dosages and for periods of time effective to result in the desired effect, which is generally thought of to be the amelioration of one or more symptoms of atherosclerosis and/or the significant reduction of the likelihood of the occurrence of one or more symptoms of atherosclerosis. Effective amounts of the pharmaceutical compositions of the invention will vary according to factors individual to the patient such age, sex, and weight of the individual. A pharmaceutical composition of the invention may be administered via any route of administration known in the art for administering therapeutic agents, e.g., oral administration, intramucosal administration, intraperitoneal injection, intravascular injection, subcutaneous injection, transcutaneous/transdermal administration, or intramuscular injection. When a peptide mimetic of the present invention is suitably protected, it may be orally administered, for example, with an inert diluent or an assimilable edible carrier. The peptide and other ingredients may also be enclosed in a hard or soft shell gelatin capsule, compressed into tablets, or incorporated directly into the individual's diet. For oral therapeutic administration, the active compound may be incorporated with excipients and used in the form of ingestible tablets, buccal tablets, troches, capsules, elixirs, solutions, gels, suspensions, syrups, wafers, and the like. Such compositions and preparations should contain at least 1% by weight of active compound. The percentage of the composition and preparations may, of course, be varied and may conveniently be between about 5 to 80% of the weight of the unit. The amount of active compound in such therapeutically useful compositions is such that a suitable dosage will be obtained. In addition, the active compound may be incorporated into sustained-release or controlled release formulations.

The concentration of peptide mimetic can but will typically be selected to provide dosages ranging from about 0.1 or 1 mg/kg/day to about 50 mg/kg/day and sometimes higher. It will be appreciated that such dosages may be varied to optimize a therapeutic regimen in a particular subject or group of subjects.

### REFERENCES

Anantharamaiah, G. M. et al, Arteriosclerosis 10 (1990) 95.
Anantharamaiah, G. M. et al, J. Lipid Research 48 (2007) 1915-1923.
Bakogianni et al., Diabetes Complications. September-October; 15(5) (2001) 265-269.
Barrans et al., Biochem Biophys Acta 1300, 73-85.
Bernal, F. et al., J. Am. Chem. Soc. 129 (2007) 2456.
Blackwell, H. E. and Grubbs, R. H., Angew. Chem. Int. Ed. 37 (1998) 3281-3284.
Bernatowicz, M. S. et al., Tetrahedron Lett. 30 (1989) 4645-4667.
Bodzioch, M. et al., Nat. Genet. 22 (1999) 347-351.
Brooks-Wilson, A. et al., Nat. Genet. 22 (1999) 336-344.
Fielding et al., J. Lipid Res 36 (1995) 211-228.
Francis, G. A., J. Clin. Investig. 96 (1995) 78-87.
Ibanez, B., et al. J. Amer. College of Cardiol., 51 (2008) 1105-1109.
Kane, J. in Fuster, V. et al. [eds.] Atherosclerosis and Coronary Artery Disease, (1996) 99.
Kurtz, M. B. et al. Antimicrob Agents Chemother 38 (1994) 2750-2757.
Miller, G. J. and Miller, N. E., Lancet 1 (1975) 16-25.
Natarajan, P. et al., J. Biol. Chem. 23 (2004) 24044-24052.
Nissen, S E et al., JAMA 290 (1993) 2292-2300.
Parolini, C et al., J. Amer. College of Cardiol., 51 (2008) 1098-1103.
Remaley, A. T. et al., Arterioscler. Throm. Vasc. Biol. 17 (1997) 1813-1821.
Remington's Pharmaceutical Sciences, 16th and 18th Eds., Mack Publishing Co., Easton, Pa. (1980 and 1990)
Rink, H., Tetrahedron Lett. 28 (1987) 3787-3789.
Segrest, J. P. et al., FEBS Lett. 38 (1974) 247-253.
Schafmeister, C. et al., J. Am. Chem. Soc. 122 (2000) 5891.
Shah P K et al., Circulation 97 (1998) 780-785.
Sole, N. A. and G. Barmy, J. Org. Chem. 57 (1992) 5399-5403.
Tall, A. & J. Breslow in Fuster, V. et al. [eds.] Atherosclerosis and Coronary Artery Disease, (1996) 106.
Walensky, L. D. et al., Science 305 (2004) 1466.
Wang, J. et al., PNAS104 (18), 7612-7616 (2007).
Wang, J. et al., J. Biol. Chem. 278 (2003) 44424-44428.

### EXAMPLES

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### Example 1

### Synthesis of the Hydrocarbon Substituents for Designing ApoA-1 Mimetic Peptides

### Synthesis of Fmoc-R5H—OH: (2R)-2-{[(9H-fluoren-9-ylmethoxy)carbonyl]amino} hept-6-enoic Acid

### Step 1: 5-iodopent-1-ene

A mixture of 5-Bromo-1-pentene (1 eq.) and NaI (3 eq.) in acetone was heated at 60° C. for 2 h. The mixture was cooled to room temperature, diluted with water and extracted with pentane. The pentane layers were combined, washed with brine, dried over Na₂SO₄, and concentrated to yield the 5-iodo-1-pentene (96%): ¹H NMR 5.75 (ddt, 1, J) 17.2, 10.4, 6.8), 5.08 (dd, 1, J) 17.2, 1.6), 5.02 (dd, 1, J) 10.4, 1.6), 3.19 (t, 2, J) 7.0), 2.17 (dt, 2, J) 6.8, 7.0), 1.91 (tt, 2, J) 7.0, 7.0).

### Step 2: (2S,5R)-2-isopropyl-3,6-dimethoxy-5-pent-4-en-1-yl-2,5-dihydropyrazine

A solution of butyllithium (1.6 N solution in hexane, 1.0 eq.) was added by syringe to a stirred solution of (2S)-2-isopropyl-3,6-dimethoxy-2,5-dihydropyrazine (1.0 eq.) in dry tetrahydrofuran at −70° C. and stirring was continued for 15 min. Then, a precooled solution of 5-iodopent-1-ene (1.0 eq.) in dry tetrahydrofuran was added and stirring was continued at −70° C. for 2-4 hours. The reaction mixture was then allowed to warm to room temperature overnight. The reaction was quenched by the addition of saturated aqueous ammonium chloride and the mixture extracted with EtOAc. The organic layer washed with brine, dried over Na₂SO₄ and evaporated in vacuo. The final crude mixture was purified by flash column chromatography, eluting with 10% EtOAc in petroleum ether.

### Step 3: methyl (2R)-2-{[(9H-fluoren-9-ylmethoxy)carbonyl]amino}hept-6-enoate

A solution of the (2S,5R)-2-isopropyl-3,6-dimethoxy-5-pent-4-en-1-yl-2,5-dihydropyrazine in a mixture of dilute aqueous hydrochloric acid (1N, 6.0 eq.) and THF (0.25M) was stirred at room temperature till the hydrolysis was complete by UPLC/MS. The reaction was quenched by the addition of 2N NaOH until neutrality and extracted with EtOAc. The organic layer was washed with brine, dried over Na₂SO₄ and evaporated in vacuo to get a mixture of methyl (2R)-2-aminohept-6-enoate and methyl L-valinate The residue was directly used in the next step without further purification to get the Fmoc-R5H-Ome (methyl (2R)-2-{[(9H-fluoren-9-ylmethoxy)carbonyl]amino}hept-6-enoate) by treating the substrate with (1.2 eq.) Fmoc-Osu in DCM at room temperature overnight. The reaction was quenched by washing the mixture with water and extracting the product in DCM. The organics was washed with brine and dried over Na₂SO₄ and evaporated in vacuo. The crude mixture obtained (Fmoc-R6H-Ome and Fmoc-S-Val-Ome) was columned on silica, eluting with from 1% to 30% MeOH, which purified the product to a limited extent (approx. 3:1, product: valine-Ome).

### Step 4: (2R)-2-{[(9H-fluoren-9-ylmethoxy)carbonyl]amino}hept-6-enoic Acid (Fmoc-R5H—OH)

The methyl (2R)-2-{[(9H-fluoren-9-ylmethoxy)carbonyl]amino}hept-6-enoate mixture was dissolved in H₂O/Dioxane (1:1) at final concentration of 0.3 M and 6 eq of HCl were added and the final mixture refluxed overnight. Final (2R)-2-{[(9H-fluoren-9-ylmethoxy)carbonyl]amino}hept-6-enoic acid was extracted into EtOAc, washed with brine and dried over Na₂SO₄. Final crude purified by flash column chromatography, eluting with from 5% to 80% CH₃CN in H₂O on KP—C18-HS 65Si (37-70 mm 300 Å) to afford to desired final Fmoc-R5H—OH.

### Example 2

### Synthesis of ApoA-1 Mimetic Peptides

The ApoA-1 mimetic peptides of the present invention (see Table 1) were synthesized by solid phase using Fmoc/tBu chemistry on a peptide synthesizer SYMPHONY (PROTEIN TECNOLOGIES, INC). For each peptide Fmoc-Linker AM-PS based resin, 1% cross-linked (BIOSEARCH TECHNOLOGIES, Inc.) and PEG-PS based resin derivatized with a modified Rink linker p-[(R,S)-α-[9H-Fluoren-9-yl-methoxyformamido]-2,4-dimethoxybenzyl]-phenoxyacetic acid (Rink, H., 1987, Tetrahedron Lett. 28:3787-3789; Bernatowicz, M. S. et al., 1989, Tetrahedron Lett. 30:4645-4667) were used. All the acylation reactions were performed for 60 minutes with a 6-fold excess of activated amino acid over the resin free amino groups following the end of peptide assembly on the synthesizer. The side chain protecting groups were: tert-butyl for Asp and Glu; tert-butyloxy carbonyl (BOC) for Lys, trityl for Asn and Gln; 2,2,4,6,7-pentamethyldihydrobenzofuran-5-sulfonyl for Arg. The proline at the N-terminal was introduced as Ac-Proline-OH by using HBTU/DIEA as coupling reagents in DMF. The hydrocarbon-substituted non-natural amino acids (R_{7H}, S_{7H}, R_{8H}, S_{8H}, S_{7H}³, R_{7H}³, S_{8H}³, R_{8H}³) were coupled manually by using HBTU/DIEA as activators and the coupling repeated if necessary. After the coupling the remainder of the synthesis was performed automatically as described above.

Alternatively, the ApoA-1 mimetic peptides were synthesized by solid phase using Fmoc/tBu chemistry on a peptide synthesizer ABI433A (APPLIED BIOSYSTEMS). For each peptide Fmoc-Linker AM-PS based resin, 1% cross-linked (BIOSEARCH TECHNOLOGIES, Inc.) was used. The acylation reactions were performed for 60 min with 4-fold excess of activated amino acid over the resin free amino groups. The amino acids were activated with equimolar amounts of HBTU (2-(1H-benzotriazole-1-yl)-1,1,3,3-tetramethyluronium hexafluorophosphate) and a 2-fold molar excess of DIEA (N,N-diisopropylethylamine) in DMF. The Proline at the N-terminal was introduced as Ac-Proline-OH by using HBTU/DIEA as coupling reagents in DMF.

At the end of the synthesis, the dry peptide-resins were individually treated with the cleavage mixture, 88% TFA, 5% phenol, 2% triisopropylsilane and 5% water (Sole, N. A. and G. Barany, 1992, J. Org. Chem. 57:5399-5403) for 1.5 hours at room temperature. In case of peptides containing olefinic-side-chain amino acids, the cleavage mixture was composed by 95% TFA and 5% water. Each resin was filtered and the solution was added to cold methyl-t-butyl ether in order to precipitate the peptide. After centrifugation, the peptide pellets were washed with fresh cold methyl-t-butyl ether to remove the organic scavengers. The process was repeated twice. Final pellets were dried, resuspended in H₂O, 20% acetonitrile, and lyophilized.

The crude peptides were purified by reverse-phase HPLC using preparative WATERS PREP LC 4000 System or GX-281 Gilson Trilution LC equipped with a REPROSIL-PUR 300 C4 column (250×20 mm, 10 μm) (DR. MAISCH GmbH) or RCM DELTA-PAK C4 cartridges (40×200 mm, 15 μm) or RCM DELTA-PAK C18 cartridges (40×200 mm, 15 μm) or ACE C18 (250×21 mm, 10 μm, 300 Å) (CPS Analitica, Milan, Italy) and using as eluents (A) 0.1% TFA in water and (B) 0.1% TFA in acetonitrile, flow rate 30 or 80 mL/min respectively.

Analytical HPLC was performed on a REPROSIL-PUR 300 C4 or C18 column (150×4.6 mm, 5 μm, 300 Å) (DR. MAISCH GmbH) or ACE C4 or C18 column (150×4.6 mm, 3 μm, 300 Å) (CPS analitica) flow rate 1 mL/min at 45° C. or Acuity UPLC BEH® C18 column (100×2.1 mm, 1.7 μm, 130 Å) (Waters) flow rate 0.4 mL/min at 45° C. The purified peptides were characterized by electrospray mass spectrometry on a Micromass LCZ platform and/or SQD Waters and/or MALDI-T of Mass Spectrometry.

The ring-closing metathesis was performed as described elsewhere (Schafmeister C. E. et all, J. Am. Chem. Soc. 2000, 122, 5891-5892), with the exception that the catalyst used was the Grubbs H catalyst. The catalyst, Ruthenium, [1,3-bis-(2,4,6-trimethylphenyl)-2-imidazolidinylidene]dichloro (phenylmethylene)(tricyclohexylphosphine), was purchased from MATERIA, Inc. (Pasadena, Calif.).

The general procedure used for the RCM for 20 mg of the peptide still bound to the solid support in a disposable fritted reaction vessel was the following:
a) The resin-peptide was swelled in 200 μL 1,2 dichloroethane which was degassed bubbling Argon directly into the reaction vessel. At this point the Grubbs II catalyst was added directly to the reaction mixture to have 10 mM final concentration. The reaction was allowed to proceed at room temperature for two hours.
b) The catalyst was filtered off. The resin washed with 1,2 dichloroethane and the catalyst was added again following the step a. The 2 hour metathesis reaction was repeated once to drive the slow metathesis reactions to completion. The resin bound peptide was then washed, dried and cleaved according to standard Fmoc peptide cleavage protocol to perform a test cleavage in order to check whether or not the RCM was complete.

The dry peptide-resins were individually treated with 20 mL of the cleavage mixture, 95% TFA and 5% water for 1.5 hours at room temperature. Each resin was filtered and washed once with neat TFA. The peptide solution was precipitated with methyl-t-butyl-ether. After centrifugation, the peptide pellets were washed with fresh cold methyl-t-butyl ether to remove the organic scavengers. The process was repeated three times. Final pellets were dried, resuspended in H₂O, 20% acetonitrile, and lyophilized. All of the metathesized peptides eluted before the unmetathesized starting material.

Crude peptides, both those unmetathesized and metathesized were purified by reverse-phase HPLC using preparative WATERS PREP LC 4000 System or GX-281 Gilson Trilution LC equipped with a REPROSIL-PUR 300 C4 (the company calls it C4, no subscript, likewise all the other ones, like C18, etc.)column (250×20 mm, 10 μm) (DR. MAISCH GmbH) or RCM DELTA-PAK C4 cartridges (40×200 mm, 15 μm) or RCM DELTA-PAK C18 cartridges (40×200 mm, 15 μm) or ACE C18 (250×21 mm, 10 μm, 300 Å) (CPS analitica) and using as eluents (A) 0.1% TFA in water and (B) 0.1% TFA in acetonitrile, flow rate 30 or 80 mL/min respectively.

Analytical HPLC was performed on a REPROSIL-PUR 300 C4 or C18 column (150×4.6 mm, 5 μm, 300 Å) (DR. MAISCH GmbH) or ACE C4 or C18 column (150×4.6 mm, 3 μm, 300 Å) (CPS Analitica) flow rate 1 mL/min at 45° C. or Acuity UPLC BEH® C18 column (100×2.1 mm, 1.7 μm, 130 Å) (Waters) flow rate 0.4 mL/min at 45° C. The purified peptides were characterized by electrospray mass spectrometry on a Micromass LCZ platform and/or SQD Waters and/or MALDI-T of Mass Spectrometry.

The ApoA-1 consensus peptide (SEQ ID No.1) was synthesized as described above with a synthetic yield of 80% The final peptide was purified by RP-HPLC by using WATERS RCM DELTA-PAK C4 cartridges with a gradient of 30-30(5 min.)-30-50 (20 min.) % B (B=0.1% TFA in CH₃CN) giving 20. % yield. The desired product was characterized by LC-MS analysis: MW 2711.18, found (MH+): 2712.68. The remaining peptides were synthesized as described above as well, either like the ApoA-1 consensus peptide or by RCM.

Another exemplary peptide is SEQ ID No11, which was synthesized by RCM as described above performing two steps of metathesis and a proper final wash of the resin after the cleavage. The synthetic yield obtained was 60%. The final peptide was purified by RP-HPLC by using a REPROSIL-PUR 300 C4 column with a gradient of 32-48% B in 25 min. (B=0.1% TFA in CH₃CN) giving 23% yield. The desired product was characterized by LC-MS analysis: 2692.24, found (MH+): 2693.65.

Another exemplary peptide was the SEQ ID No 43, which has two hydrocarbon-substituted side chain amino acids, was synthesized as described above with a synthetic yield of 73%. The final peptide was purified by RP-HPLC by using WATERS RCM DELTA PAK C4 cartridges with a gradient of 40-55% B in 25 min. (B=0.1% TFA in CH₃CN) giving 22% yield. The desired product was characterized by LC-MS analysis: MW 2741,34 found (MH+): 2742.

### Example 3

### In Vitro Functional Assay—Cholesterol Efflux

Peptides of the present invention were designed based on amphipathic properties and their abilities to solubilize phospholipids and promote cholesterol efflux. The most direct functional measurement of efficacy for the ApoA-1 peptide mimetic is its capacity to improve the efflux of cholesterol from the cell. The RAW and J774 cell lines are reliable mouse macrophage cell lines frequently used in the art for this purpose. In these cell lines, expression of the well-characterized ABCA1 cholesterol transporter protein can be upregulated by incubation with cAMP so that specific ABCA 1-stimulated efflux can be differentiated from the more non-specific mechanisms (e.g. by ABCG1 and SRB1). In the assays below RAW, J774 or comparable macrophage cell lines may be used. The RAW cell line is preferred and recited in the description below.

In the present assay, the RAW cells are plated at 1.5×10⁵ cells/200 μl/well/48-well plate in DMEM complete medium (with 10% FBS from INVITROGEN and 1% penicillin-streptomycin-glutamine from GIBCO) and ³H-cholesterol at 5 μCi/ml. After 24 hours incubation in a moist 37° C. atmosphere with 5% CO₂, the spent medium is removed, the cells are washed once with serum-free DMEM (0.1% fatty acid free bovine serum albumin replaces the FBS), and fresh serum-free medium (200 μl) is added to each well. After overnight incubation in the serum-free medium to allow the cellular cholesterol pools to equilibrate, the spent medium is removed, and fresh serum-free medium is added with/without CPT-cAMP (150 μM final). Eight-point serial titrations of the peptide/s being tested are added at this time (duplicate titrations in plain medium without cAMP and duplicate titrations in medium containing cAMP).

After 24 hours incubation in the various conditions, the medium is harvested from each well and 50 μl aliquots are counted. The cells are then lysed by adding 500 μl of HEPES/Triton lysis buffer to each well. The lysis is facilitated by freezing and thawing once. After thawing, the lysates are mixed well and 50 μl aliquots are counted.

The percent efflux of each peptide in each medium condition (±cAMP) is calculated by dividing the total counts in the medium by the sum of the total counts in the medium and lysate. The percent efflux seen with plain medium is subtracted from the percent efflux seen with the cAMP-stimulated efflux to yield the reported ABCA1-stimulated values. EC₅₀ values are determined using PRISM software (Table 2).

### Example 4

### In Vitro Functional Assay—Lipid Solubilization

As noted above, the other important parameter was phospholipid solubilization. A turbidimetric method is used to determine the solubilization capacity of peptides of the present invention. First, a three-fold serial titration of the peptide is prepared in a polypropylene v-shaped 96-well plate. The eight-point titration is done in phosphate-buffered saline (PBS, pH 7.5) or DMSO from a starting concentration that allows for addition to an optically-clear bottomed assay plate in <100 μl aliquots to yield a starting concentration of 500 μM in 200 μl final well volume. PBS is then added to each well to bring the total volume to 100 μl. Any bubbles in the wells must be dispersed to avoid erroneous results. Next, from a turbid but homogeneous emulsion of dimyristoyl phosphatidylcholine at 0.5 mg/ml in PBS, 100 μl is added to each well in the assay plate, the plate is inserted into the NEPHELOSTAR (BMG LABTECH INSTRUMENTS), and readings are taken immediately and every 15 minutes over 2 hours.

A true 0-point value is calculated by averaging the readings of the wells with phospholipid but no peptide. For each peptide concentration, a percent decrease in turbidity is calculated by subtracting the 0-point value from the corresponding 30 and 120 minute readings and dividing by that 0-point value. Finally the EC₅₀ values for both time points are determined using PRISM software (Table 2).

### Example 5

### Red Blood Cell (RBC) Lysis Assay

Increasing efficacy and decreasing toxicity are both important in drug discovery. Since the peptides of the present invention are amphipathic peptides capable of interacting with phospholipid, the ABCA1 cholesterol efflux pump and cell membrane, measurements of red blood cell (RBC) lysis and mammalian cytotoxicity are useful for defining the therapeutic windows of the peptides.

The RBC lysis assay has been described previously (Kurtz, M. B. et al. (1994); Wang, J. et al., PNAS 104 (18), 7612-7616 (2007)). Basically, human erythrocytes are collected in a vacutainer tube with EDTA from human donors. Two ml of the freshly drawn whole blood is added to 6 ml of sterile saline and gently mixed then centrifuged at 5° C. for 5 minutes at 2000 rpm. The supernatant is discarded and the human red blood cells are resuspended with 6 ml of sterile saline and centrifuged again at 5° C. for 5 minutes at 2000 rpm. This procedure is repeated 2 times. A three percent suspension of the washed red blood cells is prepared by adding 0.3 ml of the washed red blood cells to 9.7 ml of sterile saline. The final concentration of test peptide ranging 160-2.5 (or 150-2.3) μM in 100 μl sterile saline containing 3.2% Me₂SO is prepared in 96-well MICROTEST U-bottom plates (BECTON DICKINSON, BD-353227). The RBC lysis is initiated by addition of 5 μl of 3% washed human red blood cell suspension to the each well in the plates. The plates are incubated overnight at room temperature, then centrifuged at 2000 rpm for 5 minutes and read immediately. Hemolysis of red blood cells is indicated by complete or partial clearing (lysis) of the supernatants with either no or a rough RBC pellet in the bottom of the wells. No hemolysis is judged by clear supernatant with a smooth RBC pellet in the bottom of the well. The minimum lytic concentration (MLC) is defined as the lowest concentration of a test peptide to visibly produce complete or partial lysis of red blood cells. Amphotericin B is used as a positive control (MLC=2 to 4 μg/ml) and Chloramphenicol is used as a negative control (MLC=>64 μg/ml) (Table 3).

### Example 6

### Mammalian Cytotoxicity Assay

The assay is performed as previously described (Wang, J. et al. (2003)) with some modifications. Briefly, RAW cells are harvested, washed three times with MEM without phenol red (GIBCO 51200-38) and resuspended in the same medium. The cells (4×10⁴) are seeded into each well of the 96-well plates (CORNING 3904). A serial dilution of peptides is added at final concentrations of 160-2.5 μM. The plates are incubated in a moist 37° C. atmosphere with 5% CO₂. After 24 hours, 20 μl of ONE SOLUTION REAGENT (PROMEGA, G3582) is added into each well and then the plates are incubated for one hour at 37° C. and read at 490 nm. The cytotoxicity (IC₅₀) is calculated using Prism software (Table 3).

TABLE 3
RBC Lysis and Cytotoxicity of the ApoA-1 Mimetic Peptides

| | | | ABCA1- |
|---|---|---|---|
| | | | DEPENDENT |
| | | | CHOLESTEROL |
| SEQ | | | EFFLUX |
| ID NO | PEPTIDE | | EC₅₀ (MM) |
| 1 | CONS | PVLDEFREKLNEELEALKQKLK | NOT ACTIVE |
| 2 | GLA8/AIB13 | PVLDEFRGLAKLNEAIBLEALKQKLK | 44 |
| 3 | GLA8/ALA13 | PVLDEFRGLAKLNEALEALKQKLK | 142 |
| 4 | GLA15/AIB13 | PVLDEFREKLNEAIBLGLAALKQKLK | 41 |
| 5 | GLA15/ALA13 | PVLDEFREKLNEALGLAALKQKLK | 234 |
| 6 | AIB13 | PVLDEFREKLNEAIBLEALKQKLK | 6% AT 100 UM |
| 7 | AIB13/AIB16 | PVLDEFREKLNEAIBLEAIBLKQKLK | 5% AT 150 UM |
| 8 | R5H [E13-L17]R5H OPEN | PVLDEFREKLNER_{5H}LEAR_{5H}KQKLK | <3% AT 30 UM |
| 9 | R5H [K9-E13]R5H OPEN | PVLDEFRER_{5H}LNER_{5H}LEALKQKLK | 46 UM |
| 10 | S7H [F6-E13]R6H OPEN | PVLDES_{7H}REKLNER_{6H}LEALKQKLK | 1.3 UM |
| 11 | R5H [E13-L17]R5H | PVLDEFREKLNER_{5H}LEAR_{5H}KQKLK | <2% AT 30 UM |
| | CLOSED RING | (CYCLO R5H₁₃-R5H₁₇) | |
| 12 | R5H [K9-E13]R5H | PVLDEFRER_{5H}LNER_{5H}LEALKQKLK | 10 UM |
| | CLOSED RING | (CYCLO R5H₉-R5H₁₃) | |
| 13 | R6H [F6-E13]S7H OPEN | PVLDER_{6H}REKLNES_{7H}LEALKQKLK | 0.4 |
| 14 | R6H [F6-E13]S7 | PVLDER_{6H}REKLNES_{7H}LEALKQKLK | 2 |
| | CLOSED RING | (CYCLO R6H₆-S7H₁₃) | |
| 15 | S5H [K9-E13]S5H OPEN | PVLDEFRES_{5H}LNES_{5H}LEALKQKLK | 4% AT 10 UM |
| 16 | R5ME [K9-E13]R5ME | PVLDEFRER_{5ME}LNER_{5ME}LEALKQKLK | 6% AT 49 UM |
| | OPEN | | |
| 17 | CONS[R_{5H}¹³] | PVLDEFREKLNER_{5H}LEALKQKLK | 6.2% @ 100 |
| 18 | CONS[R_{6H}¹³] | PVLDEFREKLNER_{6H}LEALKQKLK | 18 |
| 19 | CONS[R_{6H}²⁰] | PVLDEFREKLNEELEALKQR_{6H}LK | 1% @ 100 |
| 20 | CONS[R_{7H}²] | PR_{7H}LDEFREKLNEELEALKQKLK | 6.3% @ 100 |
| 21 | CONS[R_{7H}¹³] | PVLDEFREKLNER_{7H}LEALKQKLK | 7.2 |
| 22 | CONS[R_{7H}²⁰] | PVLDEFREKLNEELEALKQR_{7H}LK | 1.4% @ 100 |
| 23 | CONS[S_{6H}²] | PS_{6H}LDEFREKLNEELEALKQKLK | 1% @ 100 |
| 24 | CONS[S_{6H}¹³] | PVLDEFREKLNES_{6H}LEALKQKLK | 1.4 |
| 25 | CONS[S_{6H}²⁰] | PVLDEFREKLNEELEALKQS_{6H}LK | 236 |
| 26 | CONS[S_{7H}⁶] | PVLDES_{7H}REKLNEELEALKQKLK | 4% @ 100 |
| 27 | CONS[S_{7H}⁹] | PVLDEFRES_{7H}LNEELEALKQKLK | 1.4% @ 100 |
| 28 | CONS[S_{7H}¹⁰] | PVLDEFREKS_{7H}NEELEALKQKLK | 1.4% @ 100 |
| 29 | CONS[S_{7H}¹³] | PVLDEFREKLNES_{7H}LEALKQKLK | 2 |
| 30 | CONS[S_{7H}¹⁷] | PVLDEFREKLNEELEAS_{7H}KQKLK | 1% @ 100 |
| 31 | CONS[K⁷, S_{7H}¹³] | PVLDEFKEKLNES_{7H}LEALKQKLK | 1.3 |
| 32 | CONS[S_{8H}¹³] | PVLDEFREKLNES_{8H}LEALKQKLK | 0.6 |
| 33 | CONS[E⁴, Q^{11,} S_{8H}¹³] | PVLEEFREKLQES_{8H}LEALKQKLK | 0.3 |
| 34 | CONS[S_{7H3}¹³] | PVLDEFREKLNES_{7H3}LEALKQKLK | 0.6 |
| 35 | CONS[S_{8H3}¹³] | PVLDEFREKLNES_{8H3}LEALKQKLK | 0.3 |
| 36 | CONS[E⁴, Q^{11,} S_{8H3}¹³,] | PVLEEFREKLQES_{8H3}LEALKQKLK | 0.2 |
| 37 | CONS[(RS)_{12H3}⁶] | PVLDE-(RS)_{12H3}-REKLNEELEALKQKLK | 0.2-0.4 |
| 38 | CONS[(RS)_{12H3}¹³] | PVLDEFREKLNE-(RS)_{12H3}-LEALKQKLK | 0.1-0.4 |
| 39 | CONS[(R)STYRA¹³] | PVLDEFREKLNE-(R)STYRA-LEALKQKLK | 22 |
| 40 | CONS[(S)STYRA¹³] | PVLDEFREKLNE-(S)STYRA-LEALKQKLK | 1 |
| 41 | CONS[HF-LEU^{,13}] | PVLDEFREKLNE-HFLEU-LEALKQKLK | 1.1 |
| 42 | CONS[S_{7H}^{2,13}] | PS_{7H}LDEFREKLNES_{7H}LEALKQKLK | 0.3 |
| 43 | CONS[S_{7H}^{6,13}] | PVLDES_{7H}REKLNES_{7H}LEALKQKLK | 0.3 |
| 44 | CONS[S_{7H}^{13,20}] | PVLDEFREKLNES_{7H}LEALKQS_{7H}LK | 0.6 |
| 45 | CONS[E⁴, S_{7H}^{6,13}, Q¹¹] | PVLEES_{7H}REKLQES_{7H}LEALKQKLK | 0.7 |
| 46 | CONS[GLA⁸, S_{7H}¹³] | PVLDEFR-GLA-KLNESP_{7H}LEALKQKLK | 0.9 |
| 47 | CONS[S_{7H}¹³, GLA¹⁵] | PVLDEFREKLNES_{7H}L-GLA-ALKQKLK | 0.7 |
| 48 | CON[(S)STYRA⁶, S_{7H}¹³] | PVLDE-(S)STYRA-REKLNES_{7H}LEALKQKLK | 0.2 |
| 49 | CONS[S_{8H}^{6,13}] | PVLDES_{8H}REKLNES_{8H}LEALKQKLK | 0.4 |
| 50 | CONS[S_{7H3}^{6,13}] | PVLDES_{7H3}REKLNES_{7H3}LEALKQKLK | 0.3 |
| 51 | CONS[S_{8H3}^{6,13}] | PVLDES_{8H3}REKLNES_{8H3}LEALKQKLK | 0.6 |
| 52 | CONS[S_{7H}^{2,6,13}] | PS_{7H}LDES_{7H}REKLNES_{7H}LEALKQKLK | 0.1 |
| 53 | CONS[S_{7H}^{2,13,20}] | PS_{7H}LDEFREKLNES_{7H}LEALKQS_{7H}LK | 3.2 |
| 54 | CONS[S_{7H}^{6,13,20}] | PVLDES_{7H}REKLNES_{7H}LEALKQS_{7H}LK | 0.2 |
| 55 | CONS[R_{7H}⁶, S_{7H}^{13,20}] | PVLDER_{7H}REKLNES_{7H}LEALKQS_{7H}LK | 0.1 |
| 56 | CONS[GLA¹⁵, S_{7H}^{2,13,20}] | P-S_{7H}-LDEFREKLNES_{7H}L-GLA-ALKQS_{7H}LK | 7.7 |
| 57 | CONS[GLA¹⁵, S_{7H}^{6,13,20}] | PVLDES_{7H}REKLNES_{7H}L-GLA-ALKQS_{7H}LK | 0.5 |
| 58 | CONS[R_{7H}⁶, S_{7H}^{2,13}] | PS_{7H}LDER_{7H}REKLNES_{7H}LEALKQKLK | 0.2 |
| 59 | CONS[GLA¹⁵, S_{7H}^{2,6,13}] | PS_{7H}LDES_{7H}REKLNES_{7H}L-GLA-ALKQKLK | 0.6 |
| 60 | CONS[GLA¹⁵, R_{7H}⁶, | P-S_{7H}LDER_{7H}REKLNE-S_{7H}L-GLA-ALKQKLK | 0.3 |
| | S_{7H}^{2,13}] | | |
| 61 | CONS[S_{7H}^{2,6,13,20}] | PS_{7H}LDES_{7H}REKLNES_{7H}LEALKQS_{7H}LK | 2.0 |
| 62 | CONS[S_{7H}^{2,6,13,20}, GLA¹⁵] | PS_{7H}LDES_{7H}REKLNES_{7H}L-GLA-ALKQS_{7H}LK | 2.4 |
| 63 | CONS[S_{7H}¹³]₂ | PVLDEFREKLNES_{7H}LEALKQKLKPVLDEFR | 41 |
| | | EKLNE-S_{7H}-LEALKQKLK | |
| 64 | (CONS[S_{7H}^{6,13}]-GC)₂ | (PVLDES_{7H}REKLNES_{7H}LEALKQKLKGC)₂ | 1.5 |
| 65 | CONS[S_{7H}^{6,13}]-GC-PEG₄₀ | PVLDES_{7H}REKLNES_{7H}LEALKQKLKGC(PEG₄₀) | 1% @ 100 |
| 66 | CONS[S_{7H}^{6,13}]-GC-OXA₁₂- | PVLDES_{7H}REKLNES_{7H}LEALKQKLKGC(MA- | 1.9% @ 30 |
| | CHOLESTEROL | OXA12-GLY_CHOLESTEROL) | |
| 67 | D-CONS[S_{6H}⁶, R_{7H}¹³]] | PVLDES_{6H}REKLNER_{7H}LEALKQKLK | |
| 68 | D-CONS[R_{7H}^{6,13}] | PVLDER_{7H}REKLNER_{7H}LEALKQKLK | 17.7% @ 100 |
| 69 | D-CONS[S_{7H}^{6,13}] | PVLDES_{7H}REKLNES_{7H}LEALKQKLK | 3.0 |
| 70 | D-CONS[R_{7H}²] | PR_{7H}LDEFREKLNEELEALKQKLK | 31.3% @ 100 |
| 71 | D-CONS[R_{7H}⁶] | PVLDER_{7H}REKLNEELEALKQKLK | 19% @ 100 |
| 72 | D-CONS[R_{7H}^{13]} | PVLDEFREKLNER_{7H}LEALKQKLK | 1.1 |

### Example 7

### Effects of ApoA-1 Mimetic Peptides on Reducing Atheroma in Apolipoprotein E-Deficient Mice

The ultimate goal of developing these mimetic peptides was for the acute and chronic treatment of atherosclerotic lesions in human patients. Recombinant apolipoprotein A-Mᵢₗₐₙₒ, an active mutant form of the major protein component apolipoprotein A-I in the HDL particle, has been shown to induce the regression of human coronary lesions after 5 weeks of treatment in patients with acute coronary syndromes (Nissen et al., 2003). Recombinant apolipoprotein A-I_{Milano} has also been shown to reduce lipid-rich atherosclerotic plaques in New Zealand White rabbits with induced advanced aortic lesions (Parolini et al., 2008; Ibanez et al., 2008) and to prevent the progression of aortic atherosclerosis and reduced lipid and macrophage content of plaques in apo E-deficient mice despite severe hypercholesterolemia (Shah at al., 1998). An ApoA-1 mimetic peptide (Cons[S_{7H}^{6,13}]) was administered to ApoE-deficient mice fed a normal rodent diet, at a dose of 20 mg per kg body weight, subcutaneously, once daily for 12 weeks, and found a significant reduction in atheroma volume after 5 weeks of treatment when compared to the changes in the vehicle-treated apoE-deficient mice, as assessed by magnetic resonance imaging (FIG. 11). The results demonstrated that this (Cons[S_{7H}^{6,13}]) peptide is a novel and potent reducer of atheroma volume, as compounds with a similar structure administered intraperitoneally once daily for 16 weeks, were not effective in the inhibition of lesion formation in C57BL/6J mice fed an atherogenic diet (Anantharamaiah et al., 2007).

Other embodiments are within the following claims. While several embodiments have been shown and described, various modifications may be made without departing from the spirit and scope of the present invention.

What is claimed is:

1. An ApoA-1 peptide mimetic derived from the sequence of the ApoA-1 consensus peptide by mutation of at least one amino acid to a non-natural amino acid.

2. The mimetic of claim 1, wherein said non-natural amino acid has an isobutyric side chain, a dicarboxylic acid side chain, an hydrocarbon side chain or an alkyl side chain.

3. The mimetic of claim 1, wherein said peptide mimetic exhibits an EC₅₀ between 10-0.4 μM for ABCA1-dependent cholesterol efflux.

4. The peptide of claim 1 selected from the group consisting of:
PVLDEFREKLNE-R5H-LEALKQKLK;
PVLDEFREKLNE-R6H-LEALKQKLK;
PVLDEFREKLNEELEALKQ-R6H-LK;
P-R7H-LDEFREKLNEELEALKQKLK;
PVLDEFREKLNE-R7H-LEALKQKLK;
PVLDEFREKLNEELEALKQ-R7H-LK;
P-S6H-LDEFREKLNEELEALKQKLK;
PVLDEFREKLNE-S6H-LEALKQKLK;
PVLDEFREKLNEELEALKQ-S6H-LK;
PVLDE-S7H-REKLNEELEALKQKLK;
PVLDEFRE-S7H-LNEELEALKQKLK;
PVLDEFREK-S7H-NEELEALKQKLK;
PVLDEFREKLNE-S7H-LEALKQKLK;
PVLDEFREKLNEELEA-S7H-KQKLK;
PVLDEFKEKLNES_{7H}LEALKQKLK;
PVLDEFREKLNES_{8H}LEALKQKLK;
PVLEEFREKLQES_{8H}LEALKQKLK;
PVLDEFREKLNES_{7H3}LEALKQKLK;
PVLDEFREKLNES_{8H3}LEALKQKLK;
PVLEEFREKLQES_{8H3}LEALKQKLK;
PVLDE-(RS)_{12H3}-REKLNEELEALKQKLK;
PVLDEFREKLNE-(RS)_{12H3}-LEALKQKLK;
PVLDEFREKLNE-(R)StyrA-LEALKQKLK;
PVLDEFREKLNE-(S)StyrA-LEALKQKLK;
PVLDEFREKLNE-hFLeu-LEALKQKLK;
PS_{7H}LDEFREKLNES_{7H}LEALKQKLK;
PVLDES_{7H}REKLNES_{7H}LEALKQKLK;
PVLDEFREKLNES_{7H}LEALKQS_{7H}LK;
PVLEES_{7H}REKLQES_{7H}LEALKQKLK;
PVLDEFR-Gla-KLNES_{7H}LEALKQKLK;
PVLDEFREKLNES_{7H}L-Gla-ALKQKLK;
PVLDE-(S)StyrA-REKLNES_{7H}LEALKQKLK;
PVLDES_{8H}REKLNES_{8H}LEALKQKLK;
PVLDES_{7H3}REKLNES_{7H3}LEALKQKLK;
PVLDES_{8H3}REKLNES_{8H3}LEALKQKLK;
PS_{7H}LDES_{7H}REKLNES_{7H}LEALKQKLK;
PS_{7H}LDEFREKLNES_{7H}LEALKQS_{7H}LK;
PVLDES_{7H}REKLNES_{7H}LEALKQS_{7H}LK;
PVLDER_{7H}REKLNES_{7H}LEALKQS_{7H}LK;
P-S_{7H}-LDEFREKLNES_{7H}L-Gla-ALKQS_{7H}LK;
PVLDES_{7H}REKLNES_{7H}L-Gla-ALKQS_{7H}LK;
PS_{7H}LDER_{7H}REKLNES_{7H}LEALKQKLK;
PS_{7H}LDES_{7H}REKLNES_{7H}L-Gla-ALKQKLK;
P-S_{7H}LDER_{7H}REKLNE-S_{7H}L-Gla-ALKQKLK;
PS_{7H}LDES_{7H}REKLNES_{7H}LEALKQS_{7H}LK;
PS_{7H}LDES_{7H}REKLNES_{7H}L-Gla-ALKQS_{7H}LK;
PVLDEFREKLNES_{7H}LEALKQKLKPVLDEFREKLNE-S_{7H}-
LEALKQKLK;
(PVLDES_{7H}REKLNES_{7H}LEALKQKLKGC)_{2;}
PVLDES_{7H}REKLNES_{7H}LEALKQKLKGC(PEG₄₀);
and
PVLDES_{7H}REKLNES_{7H}LEALKQKLKGC((MA-OXA12-
Gly_cholesterol).

5. A peptide mimetic of claim 1 which is selected from the group consisting of SEQ ID NO. 2-72.

6. A pharmaceutical composition comprising a peptide of claim 1.

7. A method for increasing reverse cholesterol efflux in a patient comprising the step of administering to the patient a therapeutically effective dose of a peptide of claim 1.


SEQUENCE LISTING (72 sequences)

SEQ ID NO: 1 - protein, 22 aa, Artificial Sequence
  synthetic peptide based on the ApoA-1 consensus sequence
PVLDEFREKLNEELEALKQKLK

SEQ ID NO: 2 - protein, 22 aa, Artificial Sequence
  MOD_RES 8: Xaa=gamma-carboxyglutamic acid
  MOD_RES 13: Xaa=alpha-aminoisobutyric acid
  synthetic peptide based on the ApoA-1 consensus sequence
PVLDEFRXKLNEXLEALKQKLK

SEQ ID NO: 3 - protein, 22 aa, Artificial Sequence
  MOD_RES 8: Xaa=gamma-carboxyglutamic acid
  synthetic peptide based on the ApoA-1 consensus sequence
PVLDEFRXKLNEALEALKQKLK

SEQ ID NO: 4 - protein, 22 aa, Artificial Sequence
  MOD_RES 13: Xaa= alpha-aminoisobutyric acid
  MOD_RES 15: Xaa = gamma-carboxyglutamic acid
  synthetic peptide based on the ApoA-1 consensus sequence
PVLDEFREKLNEXLXALKQKLK

SEQ ID NO: 5 - protein, 22 aa, Artificial Sequence
  MOD_RES 15: Xaa=gamma-carboxyglutamic acid
  synthetic peptide based on the ApoA-1 consensus sequence
PVLDEFREKLNEALXALKQKLK

SEQ ID NO: 6 - protein, 22 aa, Artificial Sequence
  MOD_RES 13: Xaa= alpha-aminoisobutyric acid
  synthetic peptide based on the ApoA-1 consensus sequence
PVLDEFREKLNEXLEALKQKLK

SEQ ID NO: 7 - protein, 22 aa, Artificial Sequence
  MOD_RES 13: Xaa=alpha-aminoisobutyric acid
  MOD_RES 16: Xaa = alpha-aminoisobutyric acid
  synthetic peptide based on the ApoA-1 consensus sequence
PVLDEFREKLNEXLEXLKQKLK

SEQ ID NO: 8 - protein, 22 aa, Artificial Sequence
  MOD_RES 13: Xaa = alkenyl side chain-substituted amino acid (D-glutamic acid)
  MOD_RES 17: Xaa = alkenyl side chain-substitued amino acid (D-leucine)
  synthetic peptide based on the ApoA-1 consensus sequence
PVLDEFREKLNEXLEAXKQKLK

SEQ ID NO: 9 - protein, 22 aa, Artificial Sequence
  MOD_RES 9: Xaa = alkenyl side chain-substituted amino acid (D-lysine)
  MOD_RES 13: Xaa = alkenyl side chain-substituted amino acid (D-glutamic acid)
  synthetic peptide based on the ApoA-1 consensus sequence
PVLDEFREXLNEXLEALKQKLK

SEQ ID NO: 10 - protein, 22 aa, Artificial Sequence
  MOD_RES 6: Xaa =alkenyl side chain-substituted amino acid (phenylalanine)
  MOD_RES 13: Xaa = alkenyl side chain-substituted amino acid (D-glutamic acid)
  synthetic peptide based on the ApoA-1 consensus sequence
PVLDEXREKLNEXLEALKQKLK

SEQ ID NO: 11 - protein, 22 aa, Artificial Sequence
  MOD_RES 13: Xaa = alkenyl side chain-substituted amino acid (D-glutamic acid)
  MOD_RES 17: Xaa = alkenyl side chain amino acid (D-leucine)
  synthetic peptide based on the ApoA-1 consensus sequence
PVLDEFREKLNEXLEAXKQKLK

SEQ ID NO: 12 - protein, 22 aa, Artificial Sequence
  MOD_RES 9: Xaa = alkenyl side chain-substituted amino acid (D-lysine)
  MOD_RES 13: Xaa =alkenyl side chain-substituted amino acid (D-glutamic acid)
  synthetic peptide based on the ApoA-1 consensus sequence
PVLDEFREXLNEXLEALKQKLK

SEQ ID NO: 13 - protein, 23 aa, Artificial Sequence
  MOD_RES 6: Xaa = alkenyl side chain-substituted amino acid (D-phenylalanine)
  MOD_RES 13: Xaa = alkenyl side chain-substituted amino acid (glutamic acid)
  synthetic peptide based on the ApoA-1 consensus sequence
PVLDEXHREKLNXELEALKQKLK

SEQ ID NO: 14 - protein, 23 aa, Artificial Sequence
  MOD_RES 6: Xaa = alkenyl side chain-substituted amino acid (D-phenylalanine)
  MOD_RES 13: Xaa = alkenyl side chain-substituted amino acid (glutamic acid)
  synthetic peptide based on the ApoA-1 consensus sequence
PVLDEXHREKLNXELEALKQKLK

SEQ ID NO: 15 - protein, 22 aa, Artificial Sequence
  MOD_RES 9: Xaa = alkenyl side chain-substiituted amino acid (lysine)
  MOD_RES 13: aa = alkenyl side chain-substituted amino acid (glutamic acid)
  synthetic peptide based on the ApoA-1 consensus sequence
PVLDEFREXLNEXLEALKQKLK

SEQ ID NO: 16 - protein, 22 aa, Artificial Sequence
  MOD_RES 9: Xaa = hydrocarbon side chain-substituted amino acid (D-lysine)
  MOD_RES 13: Xaa = hydrocarbon side chain-substituted amino acid (D-glutamic acid)
  synthetic peptide based on the ApoA-1 consensus sequence
PVLDEFREXLNEXLEALKQKLK

SEQ ID NO: 17 - protein, 22 aa, Artificial Sequence
  MOD_RES 13: Xaa = alkenyl side chain-substituted amino acid (D-glutamic acid)
  synthetic peptide based on the ApoA-1 consensus sequence
PVLDEFREKLNEXLEALKQKLK

SEQ ID NO: 18 - protein, 22 aa, Artificial Sequence
  MOD_RES 13: Xaa= alkenyl side chain-substituted amino acid (D-glutamic acid)
  synthetic peptide based on the ApoA-1 consensus sequence
PVLDEFRELLNEXLEALKQKLK

SEQ ID NO: 19 - protein, 23 aa, Artificial Sequence
  MOD_RES 20: Xaa= alkenyl side chain-substituted amino acid (D-lysine)
  synthetic peptide based on the ApoA-1 consensus sequence
PVLDEFREKLNEELEALKQXHLK

SEQ ID NO: 20 - protein, 22 aa, Artificial Sequence
  MOD_RES 2: Xaa= alkenyl side chain-substituted amino acid (D-valine)
  synthetic peptide based on the ApoA-1 consensus sequence
PXLDEFREKLNEELEALKQKLK

SEQ ID NO: 21 - protein, 22 aa, Artificial Sequence
  MOD_RES 13: Xaa= alkenyl side chain-substituted amino acid (D-glutamic acid)
  synthetic peptide based on the ApoA-1 consensus sequence
PVLDEFREKLNEXLEALKQKLK

SEQ ID NO: 22 - protein, 22 aa, Artificial Sequence
  MOD_RES 20: Xaa= alkenyl side chain-substituted amino acid (D-lysine)
  synthetic peptide based on the ApoA-1 consensus sequence
PVLDEFREKLNEELEALKQXLK

SEQ ID NO: 23 - protein, 22 aa, Artificial Sequence
  MOD_RES 2: Xaa=alkenyl side chain-substituted amino acid (valine)
  synthetic peptide based on the ApoA-1 consensus sequence
PXLDEFREKLNEELEALKQKLK

SEQ ID NO: 24 - protein, 22 aa, Artificial Sequence
  MOD_RES 13: Xaa=alkenyl side chain-substituted amino acid (glutamic acid)
  synthetic peptide based on the ApoA-1 consensus sequence
PVLDEFREKLNEXLEALKQKLK

SEQ ID NO: 25 - protein, 22 aa, Artificial Sequence
  MOD_RES 20: Xaa= alkenyl side chain-substituted amino acid (lysine)
  synthetic peptide based on the ApoA-1 consensus sequence
PVLDEFREKLNEELEALKQXLK

SEQ ID NO: 26 - protein, 22 aa, Artificial Sequence
  MOD_RES 6: Xaa= alkenyl side chain-substituted amino acid (phenylalanine)
  synthetic peptide based on the ApoA-1 consensus sequence
PVLDEXREKLNEELEALKQKLK

SEQ ID NO: 27 - protein, 22 aa, Artificial Sequence
  MOD_RES 9: Xaa=alkenyl side chain-substituted amino acid (lysine)
  synthetic peptide based on the ApoA-1 consensus sequence
PVLDEFREXLNEELEALKQKLK

SEQ ID NO: 28 - protein, 22 aa, Artificial Sequence
  MOD_RES 10: Xaa= alkenyl side chain-substituted amino acid (leucine)
  synthetic peptide based on the ApoA-1 consensus sequence
PVLDEFREKXNEELEALKQKLK

SEQ ID NO: 29 - protein, 22 aa, Artificial Sequence
  MOD_RES 13: Xaa= alkenyl side chain-substituted amino acid (glutamic acid)
  synthetic peptide based on the ApoA-1 consensus sequence
PVLDEFREKLNEXLEALKQKLK

SEQ ID NO: 30 - protein, 22 aa, Artificial Sequence
  MOD_RES 17: Xaa= alkenyl side chain-substituted amino acid (lysine)
  synthetic peptide based on the ApoA-1 consensus sequence
PVLDEFREKLNEELEAXKQKLK

SEQ ID NO: 31 - protein, 22 aa, Artificial Sequence
  MUTAGEN 7: arginine at Position 7 of the ApoA-1 consensus sequence mutated to lysine
  MOD_RES 13: Xaa= alkenyl side chain-substituted amino acid (glutamic acid)
  synthetic peptide based on the ApoA-1 consensus sequence
PVLDEFKEKLNEXLEALKQKLK

SEQ ID NO: 32 - protein, 22 aa, Artificial Sequence
  MOD_RES 13: Xaa= alkenyl side chain-substituted amino acid (glutamic acid)
  synthetic peptide based on the ApoA-1 consensus sequence
PVLDEFREKLNEXLEALKQKLK

SEQ ID NO: 33 - protein, 22 aa, Artificial Sequence
  MUTAGEN 4: Asp at position 4 of the ApoA-1 consensus sequence mutated to Glu
  MUTAGEN 11: Asn at position 11 of the ApoA-1 consensus sequence mutated to Gln
  MOD_RES 13: Xaa= alkenyl side chain-substituted amino acid (glutamic acid)
  synthetic peptide based on the ApoA-1 consensus sequence
PVLEEFREKLQEXLEALKQKLK

SEQ ID NO: 34 - protein, 22 aa, Artificial Sequence
  MOD_RES 13: Xaa= alkenyl side chain-substituted amino acid (glutamic acid)
  synthetic peptide based on the ApoA-1 consensus sequence
PVLDEFREKLNEXLEALKQKLK

SEQ ID NO: 35 - protein, 22 aa, Artificial Sequence
  MOD_RES 13: Xaa= alkenyl side chain-substituted amino acid (glutamic acid)
  synthetic peptide based on the ApoA-1 consensus sequence
PVLDEFREKLNEXLEALKQKLK

SEQ ID NO: 36 - protein, 22 aa, Artificial Sequence
  MUTAGEN 4: Asp at position 4 of the ApoA-1 consensus sequence mutated to Glu
  MUTAGEN 11: Asn at position 11 of the ApoA-1 consensus sequence mutated to Gln
  MOD_RES 13: Xaa= alkenyl side chain-substituted amino acid (glutamic acid)
  synthetic peptide based on the ApoA-1 consensus sequence
PVLEEFREKLQEXLEALKQKLK

SEQ ID NO: 37 - protein, 22 aa, Artificial Sequence
  MOD_RES 6: Xaa= alkyl side chain-substituted amino acid (racemic mixture of D- and L-phenylalanine)
  synthetic peptide based on the ApoA-1 consensus sequence
PVLDEXREKLNEELEALKQKLK

SEQ ID NO: 38 - protein, 22 aa, Artificial Sequence
  MOD_RES 13: Xaa= alkyl side chain-substituted amino acid (racemic mixture of D- and L-glutamic acid)
  synthetic peptide based on the ApoA-1 consensus sequence
PVLDEFREKLNEXLEALKQKLK

SEQ ID NO: 39 - protein, 22 aa, Artificial Sequence
  MOD_RES 13: Xaa= styrlalanine (R-configuration)
  synthetic peptide based on the ApoA-1 consensus sequence
PVLDEFREKLNEXLEALKQKLK

SEQ ID NO: 40 - protein, 22 aa, Artificial Sequence
  MOD_RES 13: Xaa= styrylalanine
  synthetic peptide based on the ApoA-1 consensus sequence
PVLDEFREKLNEXLEALKQKLK

SEQ ID NO: 41 - protein, 22 aa, Artificial Sequence
  MOD_RES 13: Xaa=L-hexafluoro-Leucine
  synthetic peptide based on the ApoA-1 consensus sequence
PVLDEFREKLNEXLEALKQKLK

SEQ ID NO: 42 - protein, 22 aa, Artificial Sequence
  MOD_RES 2: Xaa= alkenyl side chain-substituted amino acid (valine)
  MOD_RES 13: Xaa= alkenyl side chain-substituted amino acid (glutamic acid)
  synthetic peptide based on the ApoA-1 consensus sequence
PXLDEFREKLNEXLEALKQKLK

SEQ ID NO: 43 - protein, 22 aa, Artificial Sequence
  MOD_RES 6: Xaa= alkenyl side chain-substituted amino acid (phenylalanine)
  MOD_RES 13: Xaa= alkenyl side chain-substituted amino acid (glutamic acid)
  synthetic peptide based on the ApoA-1 consensus sequence
PVLDEXREKLNEXLEALKQKLK

SEQ ID NO: 44 - protein, 22 aa, Artificial Sequence
  MOD_RES 13: Xaa= alkenyl side chain-substituted amino acid (glutamic acid)
  MOD_RES 20: Xaa= alkenyl side chain-substituted amino acid (lysine)
  synthetic peptide based on the ApoA-1 consensus sequence
PVLDEFREKLNEXLEALKQXLK

SEQ ID NO: 45 - protein, 22 aa, Artificial Sequence
  MUTAGEN 4: D at position 4 of the consensus sequence replaced by E
  MOD_RES 6: Xaa=alkenyl side chain-substituted amino acid (phenylalanine)
  MUTAGEN 11: N at position 11 of the consensus sequence replaced by Q
  MOD_RES 13: Xaa=alkenyl side chain-substituted amino acid (glutamic acid)
  synthetic peptide based on the ApoA-1 consensus sequence
PVLEEXREKLQEXLEALKQKLK

SEQ ID NO: 46 - protein, 22 aa, Artificial Sequence
  MOD_RES 8: Xaa=gamma-carboxyglutamic acid
  MOD_RES 13: Xaa=alkenyl side chain-substituted amino acid (glutamic acid)
  synthetic peptide based on the ApoA-1 consensus sequence
PVLDEFRXKLNEXLEALKQKLK

SEQ ID NO: 47 - protein, 22 aa, Artificial Sequence
  MOD_RES 13: Xaa= alkenyl side chain-substituted amino acid (glutamic acid)
  MOD_RES 15: Xaa=gamma-carboxyglutamic acid
  synthetic peptide based on the ApoA-1 consensus sequence
PVLDEFREKLNEXLXALKQKLK

SEQ ID NO: 48 - protein, 22 aa, Artificial Sequence
  MOD_RES 6: Xaa=styrylalanine
  MOD_RES 13: Xaa=alkenyl side chain-substituted amino acid (glutamic acid)
  synthetic peptide based on the ApoA-1 consensus sequence
PVLDEXREKLNEXLEALKQKLK

SEQ ID NO: 49 - protein, 22 aa, Artificial Sequence
  MOD_RES 6: Xaa=alkenyl side chain-substituted amino acid (phenylalanine)
  MOD_RES 13: Xaa=alkenyl side chain-substituted amino acid (glutamic acid)
  synthetic peptide based on the ApoA-1 consensus sequence
PVLDEXREKLNEXLEALKQKLK

SEQ ID NO: 50 - protein, 22 aa, Artificial Sequence
  MOD_RES 6: Xaa= alkenyl side chain-substituted amino acid (phenylalanine)
  MOD_RES 13: Xaa= alkenyl side chain-substituted amino acid (glutamic acid)
  synthetic peptide based on the ApoA-1 consensus sequence
PVLDEXREKLNEXLEALKQKLK

SEQ ID NO: 51 - protein, 22 aa, Artificial Sequence
  MOD_RES 6: Xaa= alkenyl side chain-substituted amino acid (phenylalanine)
  MOD_RES 13: Xaa=alkenyl side chain-substituted amino acid (glutamic acid)
  synthetic peptide based on the ApoA-1 consensus sequence
PVLDEXREKLNEXLEALKQKLK

SEQ ID NO: 52 - protein, 22 aa, Artificial Sequence
  MOD_RES 2: Xaa=alkenyl side chain-substituted amino acid (valine)
  MOD_RES 6: Xaa=alkenyl side chain-substituted amino acid (phenylalanine)
  MOD_RES 13: Xaa=alkenyl side chain-substituted amino acid (glutamic acid)
  synthetic peptide based on the ApoA-1 consensus sequence
PXLDEXREKLNEXLEALKQKLK

SEQ ID NO: 53 - protein, 22 aa, Artificial Sequence
  MOD_RES 2: Xaa= alkenyl side chain-substituted amino acid (valine)
  MOD_RES 13: Xaa= alkenyl side chain-substituted amino acid (glutamic acid)
  MOD_RES 20: Xaa= alkenyl side chain-substituted amino acid (lysine)
  synthetic peptide based on the ApoA-1 consensus sequence
PXLDEFREKLNEXLEALKQXLK

SEQ ID NO: 54 - protein, 22 aa, Artificial Sequence
  MOD_RES 6: Xaa= alkenyl side chain-substituted amino acid (phenylalanine)
  MOD_RES 13: Xaa= alkenyl side chain-substituted amino acid (glutamic acid)
  MOD_RES 20: Xaa= alkenyl side chain-substituted amino acid (lysine)
  synthetic peptide based on the ApoA-1 consensus sequence
PVLDEXREKLNEXLEALKQXLK

SEQ ID NO: 55 - protein, 22 aa, Artificial Sequence
  MOD_RES 6: Xaa= alkenyl side chain-substituted amino acid (D-phenylalanine)
  MOD_RES 13: Xaa= alkenyl side chain-substituted amino acid (glutamic acid)
  MOD_RES 20: Xaa= alkenyl side chain-substituted amino acid (lysine)
  synthetic peptide based on the ApoA-1 consensus sequence
PVLDEXREKLNEXLEALKQXLK

SEQ ID NO: 56 - protein, 22 aa, Artificial Sequence
  MOD_RES 2: Xaa=alkenyl side chain-substituted amino acid (valine)
  MOD_RES 13: Xaa=alkenyl side chain-substituted amino acid (glutamic acid)
  MOD_RES 15: Xaa= gamma-carboxyglutamic acid
  MOD_RES 20: Xaa=alkenyl side chain-substituted amino acid (lysine)
  synthetic peptide based on the ApoA-1 consensus sequence
PXLDEFREKLNEXLXALKQXLK

SEQ ID NO: 57 - protein, 22 aa, Artificial Sequence
  MOD_RES 6: Xaa= alkenyl side chain-substituted amino acid (phenylalanine)
  MOD_RES 13: Xaa= alkenyl side chain-substituted amino acid (glutamic acid)
  MOD_RES 15: Xaa=gamma-carboxyglutamic acid
  MOD_RES 20: Xaa= alkenyl side chain-substituted amino acid (lysine)
  synthetic peptide based on the ApoA-1 consensus sequence
PVLDEXREKLNEXLXALKQXLK

SEQ ID NO: 58 - protein, 22 aa, Artificial Sequence
  MOD_RES 2: Xaa=alkenyl side chain-substituted amino acid (valine)
  MOD_RES 6: Xaa=alkenyl side chain-substituted amino acid (D-phenylalanine)
  MOD_RES 13: Xaa=alkenyl side chain-substituted amino acid (glutamic acid)
  synthetic peptide based on the ApoA-1 consensus sequence
PXLDEXREKLNEXLEALKQKLK

SEQ ID NO: 59 - protein, 22 aa, Artificial Sequence
  MOD_RES 2: Xaa=alkenyl side chain-substituted amino acid (valine)
  MOD_RES 6: Xaa=alkenyl side chain-substituted amino acid (phenylalanine)
  MOD_RES 13: Xaa=alkenyl side chain-substituted amino acid (glutamic acid)
  MOD_RES 15: Xaa=gamma-carboxyglutamic acid
  synthetic peptide based on the ApoA-1 consensus sequence
PXLDEXREKLNEXLXALKQKLK

SEQ ID NO: 60 - protein, 22 aa, Artificial Sequence
  MOD_RES 2: Xaa= alkenyl side chain-substituted amino acid (valine)
  MOD_RES 6: Xaa= alkenyl side chain-substituted amino acid (D-phenylalanine)
  MOD_RES 13: Xaa= alkenyl side chain-substituted amino acid (glutamic acid)
  MOD_RES 15: Xaa= gamma-carboxyglutamic acid
  synthetic peptide based on the ApoA-1 consensus sequence
PXLDEXREKLNEXLXALKQKLK

SEQ ID NO: 61 - protein, 22 aa, Artificial Sequence
  MOD_RES 2: Xaa= alkenyl side chain-substituted amino acid (valine)
  MOD_RES 6: Xaa= alkenyl side chain-substituted amino acid (phenylalanine)
  MOD_RES 13: Xaa= alkenyl side chain-substituted amino acid (glutamic acid)
  MOD_RES 20: Xaa= alkenyl side chain-substituted amino acid (lysine)
  synthetic peptide based on the ApoA-1 consensus sequence
PXLDEXREKLNEXLEALKQXLK

SEQ ID NO: 62 - protein, 22 aa, Artificial Sequence
  MOD_RES 2: Xaa=alkenyl side chain-substituted amino acid (valine)
  MOD_RES 6: Xaa=alkenyl side chain-substituted amino acid (phenylalanine)
  MOD_RES 13: Xaa=alkenyl side chain-substituted amino acid (glutamic acid)
  MOD_RES 15: Xaa=gamma-carboxyglutamic acid
  MOD_RES 20: Xaa=alkenyl side chain-substituted amino acid (lysine)
  synthetic peptide based on the ApoA-1 consensus sequence
PXLDEXREKLNEXLXALKQXLK

SEQ ID NO: 63 - protein, 44 aa, Artificial Sequence
  MOD_RES 13: Xaa= alkenyl side chain-substituted amino acid (glutamic acid)
  MOD_RES 35: Xaa= alkenyl side chain-substituted amino acid (glutamic acid)
  synthetic peptide based on the ApoA-1 consensus sequence
PVLDEFREKLNEXLEALKQKLKPVLDEFREKLNEXLEALKQKLK

SEQ ID NO: 64 - protein, 48 aa, Artificial Sequence
  MOD_RES 6: Xaa=alkenyl side chain -substituted amino acid (phenylalanine)
  MOD_RES 13: Xaa=alkenyl side chain -substituted amino acid (glutamic acid)
  MOD_RES 30: Xaa=alkenyl side chain -substituted amino acid (phenylalanine)
  MOD_RES 37: Xaa=alkenyl side chain -substituted amino acid (glutamic acid)
  DISULFID (23)...(24): This peptide is a dimer linked by a disulfide bridge on the terminal cysteine residues
  synthetic peptide based on the ApoA-1 consensus sequence
PVLDEXREKLNEXLEALKQKLKGCPVLDEXREKLNEXLEALKQKLKGC

SEQ ID NO: 65 - protein, 24 aa, Artificial Sequence
  MOD_RES 6: Xaa=alkenyl side chain-substituted amino acid (phenylalanine)
  MOD_RES 13: Xaa=alkenyl side chain-substituted amino acid (glutamic acid)
  MOD_RES 23: cysteine residue PEGylated via the side chain thiol with branched mPEG 40 kDa
  synthetic peptide based on the ApoA-1 consensus sequence
PVLDEXREKLNEXLEALKQKLKGC

SEQ ID NO: 66 - protein, 24 aa, Artificial Sequence
  MOD_RES 6: Xaa= alkenyl side chain-substituted amino acid (phenylalanine)
  MOD_RES 13: Xaa= alkenyl side chain-substituted amino acid (glutamic acid)
  LIPID 23: Cysteine residue is esterified to a glycine which has a PEG spacer connecting to the cholesterol moiety
  synthetic peptide based on the ApoA-1 consensus sequence
PVLDEXREKLNEXLEALKQKLKGC

SEQ ID NO: 67 - protein, 22 aa, Artificial Sequence
  MOD_RES 6: Xaa= alkenyl side chain-substituted amino acid (L-phenylalanine)
  MOD_RES 13: Xaa= alkenyl side chain-substituted amino acid (D-glutamic acid)
  MOD_RES (1)...(22): Except for position 6, entire peptide is in the D-conformation
  synthetic peptide based on the ApoA-1 consensus sequence
PVLDEXREKLNEXLEALKQKLK

SEQ ID NO: 68 - protein, 22 aa, Artificial Sequence
  MOD_RES 6: Xaa= alkenyl side chain-substituted amino acid (D-phenylalanine)
  MOD_RES 13: Xaa= alkenyl side chain-substituted amino acid (D-glutamic acid)
  MOD_RES (1)...(22): Entire peptide is in the D-conformation
  synthetic peptide based on the ApoA-1 consensus sequence
PVLDEXREKLNEXLEALKQKLK

SEQ ID NO: 69 - protein, 22 aa, Artificial Sequence
  MOD_RES 6: Xaa= alkenyl side chain-substituted amino acid (L-phenylalanine)
  MOD_RES 13: Xaa= alkenyl side chain-substituted amino acid (L-glutamic acid)
  MOD_RES (1)...(22): Except for positions 6 and 13, the entire peptide is in the D-conformation
  synthetic peptide based on the ApoA-1 consensus sequence
PVLDEXREKLNEXLEALKQKLK

SEQ ID NO: 70 - protein, 21 aa, Artificial Sequence
  MOD_RES (1)...(21): Entire peptide is in the D-conformation
  MOD_RES 2: Xaa= alkenyl side chain-substituted amino acid (D-valine)
  synthetic peptide based on the ApoA-1 consensus sequence
PXDEFREKLNEELEALKQKLK

SEQ ID NO: 71 - protein, 22 aa, Artificial Sequence
  MOD_RES (1)...(22): Entire peptide is in the D-conformation
  MOD_RES 6: Xaa= alkenyl side chain-substituted amino acid (D-phenylalanine)
  synthetic peptide based on the ApoA-1 consensus sequence
PVLDEXREKLNEELEALKQKLK

SEQ ID NO: 72 - protein, 22 aa, Artificial Sequence
  MOD_RES (1)...(22): Entire peptide is in the D-conformation
  MOD_RES 13: Xaa= alkenyl side chain-substituted amino acid (D-glutamic acid)
  synthetic peptide based on the ApoA-1 consensus sequence
PVLDEFREKLNEXLEALKQKLK